(19) United States
(12) Patent Application Publication
(10) Pub. No.: US 2026/0192018 A1
(43) Pub. Date: Jul. 9, 2026

(54) COMPOSITION AND FAT GRAFTING USING THE SAME

(71) Applicants: The Johns Hopkins University, Baltimore, MD (US); LIFESPROUT, INC., Baltimore, MD (US)

(72) Inventors: Hai-Quan Mao, Baltimore, MD (US); Sashank Reddy, Baltimore, MD (US); Yueh-Hsun Kevin Yang, Baltimore, MD (US); Jiayuan Kong, Baltimore, MD (US); Zhicheng Yao, Baltimore, MD (US); Russell Martin, Baltimore, MD (US)

(21) Appl. No.: 19/128,865

(22) PCT Filed: Nov. 10, 2023
(86) PCT No.: PCT/US23/37155

§ 371 Date: May 9, 2025

Related U.S. Application Data
(60) Provisional application No. 63/424,345, filed on Nov. 10, 2022.

(51) Int. Cl. A61L 27/36; A61L 27/18; A61L 27/20; A61L 27/24; A61L 27/52; C08L 5/08; C08L 67/04; C08L 89/06

(52) U.S. Cl. CPC A61L 27/3604; A61L 27/18; A61L 27/20; A61L 27/24; A61L 27/3687; A61L 27/52; C08L 5/08; C08L 67/04; C08L 89/06; A61L 2400/06; A61L 2430/34

(57) ABSTRACT

Provided herein, inter alia, are soft tissue devices including hydrogel composites, compositions, and use for fat grafting and regenerating soft tissue.

The present application claims the benefit of priority of U.S. provisional application No. 63/424,345 filed Nov. 10, 2022, which is incorporated by reference herein in its entirety.

### TECHNICAL FIELD

Provided herein, inter alia, are soft tissue devices including hydrogel composites, compositions, and use for fat grafting and regenerating soft tissue.

### BACKGROUND

Soft tissue defects resulting from trauma, oncologic resection, or congenital malformation have been treated using fat grafting. For example, in 2019, there were 600,000 fat grafting procedures in the face worldwide, and substantially more fat grafting procedures in other anatomical regions. The tissue regrowth may need a suitable matrix for cells to attach, migrate, proliferate, differentiation, and organize into new tissue. For example, native extracellular matrix (ECM) has been used at the repair site. However, there is no current material that can endure mechanical and structural challenges of cells during the restoration.

### SUMMARY

Provided herein, inter alia, composition for administration (soft tissue devices) including hydrogel composites for fat grafting and regenerating soft tissue.

In one aspect, we now provide composition for administration comprising a) fat cells or tissue; and b) non-spherical microbeads comprising a hydrogel composite, the hydrogel composite comprising a hyaluronic acid component covalently linked to a fiber component, wherein a a weight ratio between the hyaluronic acid component to the fiber component ranges from 1:100 to about 100:1.

In an aspect, provided is a soft tissue device including a biologically active material, and non-spherical microbeads comprising a hydrogel composite. In one aspect, the hydrogel composite comprises a functionalized hyaluronic acid network and an associated fiber or scaffold component. In a particular aspect, the hydrogel composition suitably comprises 1) a fiber or scaffold component; 2) hyaluronic acid including functionalized hyaluronic acid; and preferably 3) a crosslinking component.

In a further aspect, a composition for administration or a soft tissue device is provided, the composition or device, comprising: a non-spherical microbeads comprising a hydrogel composite, the hydrogel composite comprising a hyaluronic acid component covalently linked to a fiber component, and b) a population of adipose cells, autologous adipose cells, allogenic cells, genetically modified allogenic cells, adipose stromal vascular fraction, adipose tissue, autologous adipose tissue, lipoaspirate, a derivative thereof, or a combination thereof, wherein the composition soft tissue device comprises the non-spherical microbeads in a volume of about 25% to 75% of the total volume of the soft tissue device; and a weight ratio between the hyaluronic acid component to the fiber component ranges from 1:100 to about 100:1.

In certain aspects, the fat cells of fat tissue may be in the form of fat particles. As referred to herein, fat cells or fat tissue or other similar term includes adipose cells or tissue. In some embodiments, the fat cells or tissue or adipose tissue is lipoaspirate. In some embodiments, the fat cells, fat tissue or adipose tissue is autologous. Fat particules may be present as an admixture with extracellular matrix proteins (ECM) material such as a collagen material.

In an embodiment, suitably a ratio of the mean size of the non-spherical microbeads to a mean size of the fat particles ranges from about 10:1 to 1:10. In an embodiment, suitably a ratio of the mean size of the non-spherical microbeads to a mean size of the fat particles ranges from about 2:8 to 8:2. In an embodiment, suitably a ratio of the mean size of the non-spherical microbeads to a mean size of the fat particles ranges from about 3:7 to 7:3. In an embodiment, suitably of the mean size of the non-spherical microbeads to a mean size of the fat particles ranges from about 4:6 to 6:4. In an embodiment, suitably a mean size of the non-spherical microbeads is within 5 or 10% of a mean size of the fat particles. In aspects, fat particles are suitably below one millimeter in size (longest dimension), e.g. up to or less than 0.9, 0.8, 0.7, 0.6, 0.5, 0.4, 0.3 0.3 or 0.1 millimeters in a longest dimension.

In an embodiment, the fiber component comprises non-woven polymeric fiber. In certain embodiments, the polymeric fiber includes a polycaprolactone fiber such as an electrospun polycaprolactone fiber. Optionally, the polymeric fiber includes a synthetic polymeric material comprising for example a poly(lactic-co-glycolic acid), a polylactic acid, and/or a polycaprolactone, or a combination thereof. In one embodiment, the complex is formulated to be substantially biocompatible. Optionally, the polymeric fiber includes a biological polymeric material that includes a silk, a collagen, a chitosan, and/or a mimetic or combination thereof. In one aspect, collagen-based peptides e.g. with or without hydroxyproline residues, including recombinant peptides may be a suitable material of a fiber element.

In a preferred aspect, the hydrogel composition includes a functionalized hyaluronic acid network covalently linked to a plurality of polycaprolactone nanofibers.

In a preferred aspect, the hydrogel composition includes a functionalized hyaluronic acid network covalently linked to a plurality of collagen nanofibers. In embodiments, collagen nanofibers suitably may be naturally obtained or synthesized. In certain embodiments, the collagen nanofiber includes a type I bovine collagen nanofiber or fragments thereof. In certain embodiments, the collagen nanofiber includes collagen mimetics such as collagen-like peptide sequences. In certain embodiments, the collagen nanofibers may be obtained from natural sources, or may be fabricated or prepared from a composition (resin composition) including collagen. For example, the collagen nanofiber may be formed by electrospinning, centrifugal spinning, blow spinning, or combinations thereof. Particularly, collagen nanofibers are preferably prepared by electrospinning.

In certain preferred aspects, the mean size of the non-spherical microbeads is within the range of about 50 micrometers to about 300 or 400 micrometers along the longest dimension, including where the the mean size of the non-spherical microbeads along a longest dimension is up to about 50, 60, 70, 80, 90, 100, 120, 150, 180, 200, 220, 250, 280, 300, 320, 350 or 400 micrometers along the longest dimension.

Preferably, the biologically active material (such as fat cells or tissue) that is present with non-spherical microbeads comprising a hydrogel composite includes a population of adipose cells, autologous adipose cells, allogenic cells, genetically modified allogenic cells, stem cells, mesenchymal stem cells, genetically modified stem cells, genetically modified allogenic induced pluripotent stem (iPS) cells, genetically modified hypoimmunogenic pluripotent stem cells, adipose stromal vascular fraction, adipose tissue, autologous adipose tissue, lipoaspirate, a derivative thereof, or a combination thereof,

Preferably, the soft tissue devise includes the non-spherical microbeads in a volume of about 25% to 75% of the total volume of the soft tissue device, including where the non-spherical microbeads in present in a volume of at least about 25% and up 30%, 35%, 40%, 50%, 55%, 60%, or 70% of the total volume of the soft tissue device.

In one embodiment, in a composite that comprises non-spherical microbeads, the composite may comprise a crosslinking agent for example present at a concentration from about 1 mg/mL to about 25 mg/mL, preferably wherein the mean size of the microbeads is within the range of about 50 micrometers to about 300 micrometers along the longest dimension, suitably wherein the microbeads are pre-reacted (e.g. the crosslinking agent has reacted with one or more other components on the microbeads), and/or preferably wherein the microbeads are substantially stable at room temperature for at least about 6 months.

In an aspect, provided is a method of fat grafting in a subject. The method comprises administering an effective amount of a composition (soft tissue device) as disclosed herein to a subject in need thereof. In one aspect, the composition suitably is implanted into a target tissue of the subject. In one aspect, the composition suitably is injected into a target tissue of the subject.

Also, in an aspect, provided is a method for performing a cosmetic procedure or a reconstructive procedure or reducing or reversing a tissue defect resulting from trauma, surgical intervention, or an age-associated disease, disorder or condition.

The method comprises administering an effective amount of a composition (soft tissue device) as disclosed herein to a subject in need thereof. In one aspect, the composition suitably is implanted into a target tissue of the subject. In one aspect, the composition suitably is injected into a target tissue of the subject.

Further provided is a kit for preparation of the injecting the soft tissue device as described herein for administration into a target tissue of a subject. The kit includes (i) a first syringe comprising the microbeads; and (ii) a second syringe comprising the biologically active material which may comprise fat cells or fat tissue. The kit further includes (iii) a luer-luer union connector with an orifice allowing the passage of the microbeads and biological active material for mixing prior to injection or implantation. Preferably, the microbeads are gelated or cured before the biological active material is added to the microbeads.

Microbeads and fat particle sizes can be readily determined by known methods, including optical methods of measuring bead size and fat particle size and histology measurements for fat particle size. In particular, dynamic light scattering and laser particle analyzers can be used to measure size of microbeads and fat particles as referred to herein.

Fat particles as referred to herein suitably may be prepared by a variety of processes, including mechanical treatment of fat cells or tissue. Exemplary fat particle formation is set forth in Example 3 which follows, and includes forming an admixture of fat tissue/cells, casting the admixture to a film layer (optionally cross-linking) followed by particle formation of film layer such as with mechanical treatment (e.g. grinding by e.g. pestal) or cryogrinding. Alternatively, fat obtained from a mammal (e.g., human, rodent such as rat or other mammal) e.g. by using aspirator (e.g., vacuum pump) and the obtained fat may be harvested using homogenizer to provide desired fat particles as referred to herein. The collected fat samples may be filtered and the filtration may be controlled based on the desired particle size. Various commercially available apparatus such as REVOLVE System™ (Abbvie) may be used for preparing the fat particles. In a still further exemplary approach to provide fat particles, fat tissue can be surgically harvested en bloc from a donor site (either from the same animal/person or from a donor animal/person), then mechanically macerated to generate fat particles with intact cells in extracellular matrix. As discussed above, in certain embodiments, fat particles may comprise fat cells/tissue together with an extracellular matrix (ECM) material such as a collagen material.

Other aspects of the invention are disclosed infra.

### BRIEF DESCRIPTION OF THE DRAWINGS

FIG. 1A shows an exemplary bead including a nanofiber (101), a crosslinking agent (102) and a hyaluronic acid (103).

FIG. 1B shows an exemplary soft tissue device including an exemplary bead and a biologically active material, e.g., fat.

FIG. 2 shows a scheme to react a modified hyaluronic acid, surface-functionalized nanofibers and a crosslinker (polyethylene glycol dithiol) together to form a composite hydrogel structure with covalent linkage between fiber and hydrogel phases.

FIG. 3 shows a scheme to modify a fiber surface to add functional groups for covalent linkage to the hydrogel phase according an exemplary embodiment of the disclosure.

FIG. 4 (includes FIGS. 4A-4C) shows preparation of Nanofiber-Hydrogel Composite (NHC)-Fat Mixtures for Fat Grafting made with polycaprolactone (PCL) nanofibers from Example 2. FIG. 4A: A representative image of the processed fat fragments. FIG. 4B: The NHC and the processed fat mixed at different volumetric ratios. Scale bar: 1 cm. FIG. 4C: Injection schemes for fat grafting experiments of Example 4.

FIG. 5A shows storage Moduli of the Prepared NHC-Fat Grafting Materials from Example 2. Comparable G′ values of around 300 Pa were identified across different NHC-Fat combinations and no statistically significant difference was detected. n=12; p>0.05. FIG. 5B shows tan delta values of the NHC-Fat combinations.

FIG. 6 shows distribution of the NHC and the Processed Fat at POD 0 Verified by H&E Staining from Example 4. The processed fat fragments were well mixed with the NHC leading to a uniform distribution of the two components within each type of graft. The NHC is shown in a light-gray color while the processed fat contains an ECM stained in a pink color and adipocytes with a vacuole structure. Scale bars in the overall images and in the extracted images are 2.5 mm and 500 μm, respectively.

FIG. 7 (includes FIGS. 7A-7C) shows volume and shape retention of injected grafts from Example 4. FIG. 7A: MRI images were taken every 15 days from POD 0 to POD 90 to estimate the graft volume shown in (B). The shape of the graft became flatter over time in most of the groups. FIG. 7B: The 100% NHC and the 50% NHC-50% Fat groups resulted in better volume retention than the other three groups by POD 90. n=4. FIG. 7C: Representative gross images of each type of graft at PODs 0, 30 and 90. It was difficult to visualize the 100% fat grafts by POD 90. Scale bar: 1 cm.

FIG. 8 shows morphological analysis of injected grafts from Example 4. The pure NHC graft remodeled into a capsule-like construct primarily composed of soft tissue. The 75% NHC-25% Fat grafts experienced early tissue remodeling into neo-soft tissue and lost most of the co-injected fat by POD 30. Vacuoles were identified in the grafts composed of 50% fat fragments or more at POD 30, but only the 50% NHC-50% Fat group preserved healthy vacuoles at POD 90 while enlarged vacuoles were observed in both 25% NHC-75% Fat and 100% Fat samples. Nuclei, ECM or cytoplasm, and the NHC are shown in purple, pink, and light gray, respectively. Scale bars in the overall images and in the extracted images are 2.5 mm and 500 μm, respectively.

FIG. 9 shows detection of viable adipocytes in injected grafts from Example 4. The specimens were labeled for perilipin to visualize viable adipocytes. A dying adipocyte is defined as a cell that has an enlarged vacuole with no or fading perilipin staining and is usually surrounded by infiltrating host cells (DAPI). Abundant perilipin-expressing cells were observed in the 50% NHC-50% Fat group whereas most of adipocytes in 25% NHC-75% Fat and 100% Fat groups were non-viable. Few viable adipocytes were located at the periphery of the remodeled soft tissue in 100% NHC and 75% NHC-25% Fat groups at POD 90. Nuclei and perilipin are shown in blue and red, respectively. Scale bar: 200 μm.

FIG. 10 shows formation of blood vessels in injected grafts from Example 4. The specimens were co-labeled for αSMA and RECA-1 to visualize blood vessels. Increased vascularization was observed in the 50% NHC-50% Fat group whereas the pure fat grafts were poorly vascularized. While larger vessels were located in the peripheral region of the construct in 100% NHC, 75% NHC-25% Fat, and 25% NHC-75% Fat groups at POD 90, some small vessels were also observed toward the center of the sample. Nuclei, αSMA, and RECA-1 are shown in blue, red, and green, respectively. Scale bar: 200 μm.

FIG. 11 shows a scheme of an exemplary method of extracting fat and mix them with NHC.

FIG. 12 (includes FIGS. 12A-12C) shows the volume retention of injected grafts from Example 7. FIG. 12A: The composition of different fat-NHC (with collagen fibers, prepared in Example 6) grafts. FIG. 12B: Gross images of each injected graft after 1 month. FIG. 12C: Volumetric measurements based on gross images for each injected graft.

FIG. 13 shows morphological analysis of injected grafts based on hemoxylin and eosin staining at POD 30 from Example 7. The samples were stained with hemoxylin and eosin. Nuclei, cytoplasm, and the NHC are shown in purple, pink and light gray, respectively.

FIG. 14 shows detection of viable adipocyte in injected collagen based NHC-fat grafts from Example 7. The samples were stained for perilipin to visualize viable adipocytes. Red arrows mark healthy adipocytes that express strong perilipin signal. Yellow arrows mark non-viable adipocytes with enlarged vacuoles in the absence of perilipin expression. 75% Fat-25% NHC and 50% Fat-50% NHC grafts showed a superior adipocyte survival rate at POD 30.

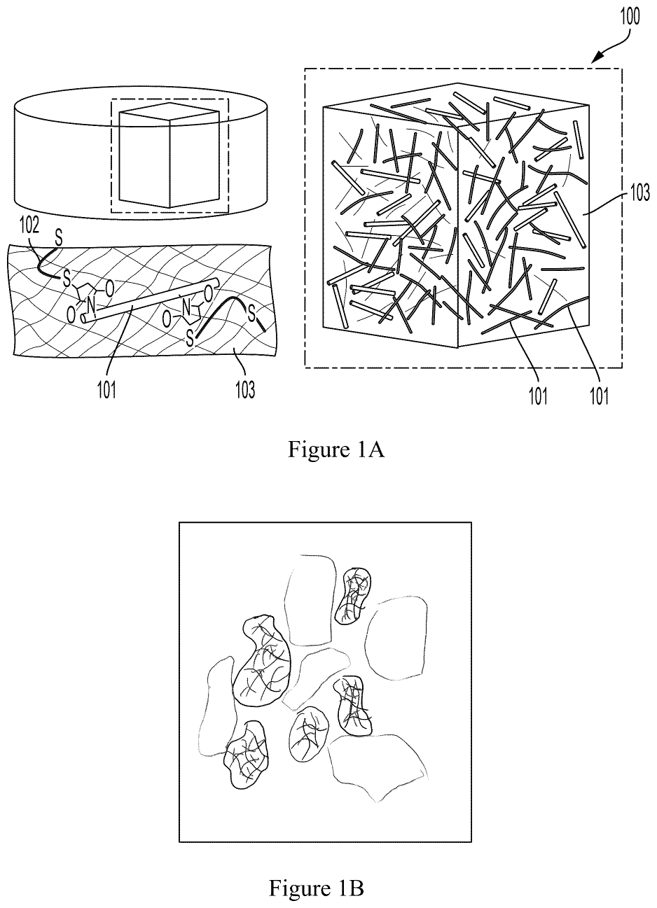

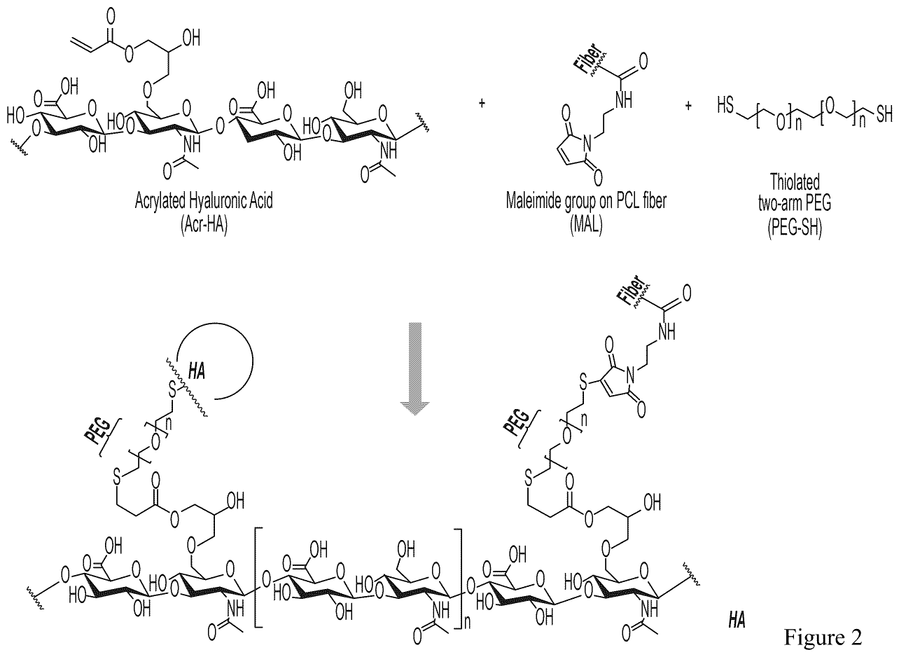

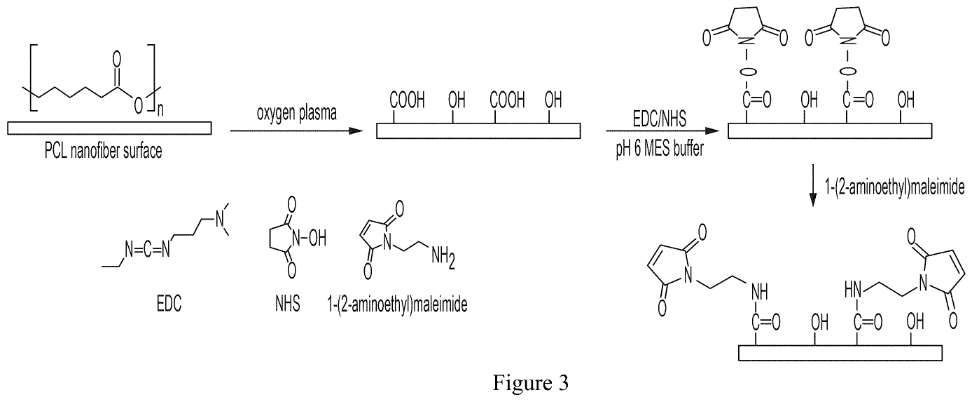

### DETAILED DESCRIPTION

The following detailed description, given by way of example, but not intended to limit the invention solely to the specific embodiments described, may best be understood in conjunction with the accompanying drawings.

The present invention relates to pre-reacted, beaded composite materials comprising a hydrogel and a nanostructure for use in methods for reconstruction of soft tissue. The invention also relates to a soft tissue device comprising beaded composite materials for cell and tissue delivery for cosmetic, reconstructive, and cellular therapies.

The invention also relates to composite materials that can recruit, capture, encapsulate, associate, and/or embed specific tissue constituents including but not limited to adipocytes, other mesenchymal cells, or mesenchymal stem cells. The invention further relates to composite materials that can recruit, capture, encapsulate, associate, and/or embed specific tissues including but not limited to adipose tissues. The invention also relates to methods for repairing or reconstructing a soft tissue injury using a composition comprising a scaffold complex (such as soft tissue device) comprising a biomaterial covalently linked to a biodegradable fiber. The invention in other aspects also relates to a method of fabricating a composition for use in soft tissue reconstruction where the composition comprises a hydrogel and a nanostructure disposed therein. The invention in particular aspects also relates to a method of fabricating a composition for use in cell and tissue delivery for cosmetic, reconstructive, and cellular therapies.

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

Although any methods and materials similar or equivalent to those described herein can also be used in the practice or testing of the present invention, the preferred methods and materials are now described. All publications mentioned herein are incorporated herein by reference to disclose and described the methods and/or materials in connection with which the publications are cited.

Unless defined otherwise, all technical and scientific terms used herein have the meaning commonly understood by a person skilled in the art to which this invention belongs. The following references, the entire disclosures of which are incorporated herein by reference, provide one of skill with a general definition of many of the terms (unless defined otherwise herein) used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, the Harper Collins Dictionary of Biology (1991). Generally, the procedures of molecular biology methods described or inherent herein and the like are common methods used in the art. Such standard techniques can be found in reference manuals such as for example Sambrook et al., (2000, Molecular Cloning—A Laboratory Manual, Third Edition, Cold Spring Harbor Laboratories); and Ausubel et al., (1994, Current Protocols in Molecular Biology, John Wiley & Sons, New-York).

The following terms may have meanings ascribed to them below, unless specified otherwise. However, it should be understood that other meanings that are known or understood by those having ordinary skill in the art are also possible, and within the scope of the present invention. All publications, patent applications, patents, and other references mentioned herein are incorporated by reference in their entirety. In the case of conflict, the present specification, including definitions, will control. In addition, the materials, methods, and examples are illustrative only and not intended to be limiting.

### Definitions

The term “a” and “an” refers to one or to more than one (i.e., to at least one) of the grammatical object of the article. By way of example, “an element” means one element or more than one element.

As used herein, “about” can mean plus or minus less than 1 or 1, 2, 3, 4, 5, 6, 7, 8, 9, 10, 11, 12, 13, 14, 15, 16, 17, 18, 19, 20, 25, 30, or greater than 30 percent, depending upon the situation and known or knowable by one skilled in the art.

As used herein the specification, “subject” or “subjects” or “individuals” may include, but are not limited to, mammals such as humans or non-human mammals, e.g., domesticated, agricultural or wild, animals, as well as birds, and aquatic animals. In certain embodiments, the subject is a human patient or an animal subjected to medical treatment.

As used herein, the term “hydrogel” is a type of “gel,” and refers to a water-swellable polymeric matrix, consisting of a three-dimensional network of macromolecules (e.g., hydrophilic polymers, hydrophobic polymers, blends thereof) held together by covalent or non-covalent crosslinks that can absorb a substantial amount of water (e.g., 50%, 60% 70%, 80%, 90%, 95%, 96%, 97%, 98%, 99% or greater than 99% per unit of non-water molecule) to form an elastic gel. The hydrogel may contain “water-swellable” polymer is one that absorbs an amount of water greater than at least 50% of its own weight, upon immersion in an aqueous medium. The polymeric matrix may be formed of any suitable synthetic or naturally occurring polymer material. As used herein, the term “gel” refers to a solid three-dimensional network that spans the volume of a liquid medium and ensnares it through surface tension effects. This internal network structure may result from physical bonds (physical gels) or chemical bonds (chemical gels), as well as crystallites or other junctions that remain intact within the extending fluid. Virtually any fluid can be used as an extender including water (hydrogels), oil, and air (aerogel). Both by weight and volume, gels are mostly fluid in composition and thus exhibit densities similar to those of their constituent liquids. A hydrogel is a type of gel that uses water as a liquid medium.

In certain embodiments, the hydrogel is a composite or composite material. The term “composite” as used herein includes any association, bonding or attachments of two or more components. In some embodiments, the “hydrogel composite” as used herein include at least a polymeric fiber and a hydrogel material. The hydrogel composite contains the polymeric fiber (e.g., polycaprolactone) and hydrogel material (e.g., hyaluronic acid (HA)).

A term “functional network” as used herein means that the interactions between components results in a chemical, biochemical, biophysical, physical, or physiological benefit. In addition, a functional network may include additional components, including cells, biological materials (e.g., polypeptides, nucleic acids, lipids, carbohydrates), therapeutic compounds, synthetic molecules, and the like. In certain embodiments, the scaffold complex promotes tissue growth and cell infiltration when implanted into a target tissue present in a human subject.

The term “nanofiber” as used herein refers to a fibrous material having at least one dimension (e.g., length, or width) less than about 999 nm, less than about 900 nm, less than about 800 nm, less than about 700 nm, less than about 600 nm, less than about 500 nm, less than about 400 nm, less than about 300 nm, less than about 200 nm, less than about 100 nm, less than about 50 nm, or less than about 10 nm. In some embodiments, the nanofibers may have a length less than about 999 nm, less than about 900 nm, less than about 800 nm, less than about 700 nm, less than about 600 nm, less than about 500 nm, less than about 400 nm, less than about 300 nm, less than about 200 nm, less than about 100 nm, less than about 50 nm, or less than about 10 nm. In some embodiments, the nanofibers may have a width less than about 999 nm, less than about 900 nm, less than about 800 nm, less than about 700 nm, less than about 600 nm, less than about 500 nm, less than about 400 nm, less than about 300 nm, less than about 200 nm, less than about 100 nm, less than about 50 nm, or less than about 10 nm. The term “nanofiber” s used herein also includes microfibers e.g. with at least one dimension less than 10 microns in size, since they can have surface features under 1 micron in size and can be made with similar methods and materials.

The term “microfiber” as used herein includes a fibrous material having at least one dimension (e.g., length, or width) less than about 10 microns.

The term “nanofiber-hydrogel composite” as used herein refers to a composite including at least nanofibers (e.g., polymeric fibers) and hydrogel (e.g., HA), which form functional networks. In addition, the “nanofiber-hydrogel composite,” “hydrogel composite,” “composite” or “complex” as used herein are interchangeably used referring to such composite including at least nanofibers (e.g., polymeric fibers) and hydrogel (e.g., HA).

The term “crosslinked” herein refers to a composition containing intramolecular and/or intermolecular crosslinks, whether arising through covalent or noncovalent bonding, and may be direct or include a cross-linker. “Noncovalent” bonding includes both hydrogen bonding and electrostatic (ionic) bonding.

The term “polymer” includes linear and branched polymer structures, and also encompasses crosslinked polymers as well as copolymers (which may or may not be crosslinked), thus including block copolymers, alternating copolymers, random copolymers, and the like. Those compounds referred to herein as “oligomers” are polymers having a molecular weight below about 1000 Da, preferably below about 800 Da. Polymers and oligomers may be naturally occurring or obtained from synthetic sources.

As used herein, the term “biomaterial” means an organic material that has been engineered to interact with biological systems. In some embodiments of the invention, a biomaterial is a hydrogel. In some embodiments, biomaterial is a bacterially derived hyaluronic acid (HA).

As used herein, the term “biodegradable” refers to a material that can be broken down by biological means in a subject.

As used herein, the term “implantable” means able to be formulated for implantation via a syringe to a subject.

As used herein, the term “soft tissue” refers to tissues that connect, support, or surround other structures and organs of the body. Soft tissue includes muscles, tendons, ligaments, fascia, nerves, fibrous tissues, fat, blood vessels, and synovial membranes.

As used herein, the term “stable” refers to a material that does not degrade at room temperature.

As used herein, the term “autologous” refers to any material derived from the same individual to whom it is later to be re-introduced into the individual.

As used herein, the term “allogeneic” or, alternatively, “allogenic,” refers to any material derived from a different animal of the same species or different patient as the individual to whom the material is introduced.

As used herein, the term “lyophilized” refers to a material after undergoing a lyophilization, which is a process used for preserving materials by removing the water from the material, which involves first freezing the material and then drying it, under a vacuum, at very low temperatures.

As used herein, the term “functionalized” refers to a material that is uniformly or non-uniformly modified so as to have a functional chemical moiety associated therewith (e.g., chemically modified). In some cases, functional chemical moiety is capable of reacting to permit the formation of a covalent or non-covalent bond. In some cases, functional chemical moiety can provide the material improved properties.

### Nanofiber-Hydrogel Composite

Provided is a “nanofiber-hydrogel composite,” “hydrogel composite,” or “composite” that is formed by combining hydrogel materials or other biomaterials with polymeric nanofibers. The composite may be formulated such that the density, ratio of gel to fibers, and other properties are variable, while maintaining sufficient porosity and strength.

A ratio of polymeric nanofibers to hydrogel material can be determined my any means known in the art. For example, the ratio of polymeric fiber to hydrogel material is from about 1:100 to about 100:1 on a component-mass basis, such as about 1:50 to about 50:1, or 1:10 to about 10:1, such as 1:5 to about 5:1, such as about 1:3 to about 3:1. The ratio of polymeric fiber to hydrogel material is also provided as a concentration basis, e.g., a given weight of polymeric fiber per volume of hydrogel material. For example, the concentration is from about 1 to 50 mg/mL. The hydrogel material is generally disposed on the polymer fiber, such as being bonded to the outer surface (or an outer surface, depending upon the composition and shape) of the polymer fiber. In certain embodiments, the plurality of polymer fibers (e.g., polycaprolactone nanofibers) are present on the surface of the hydrogel composite. In certain embodiments, the plurality of polymer fibers (e.g., polycaprolactone nanofibers) are present on the surface of the hydrogel composite beads (e.g., microbeads).

The composite may contain a plurality of pores present on or within a surface of the composite. The presence, size, distribution, frequency, and other parameters of the pores can be modulated during the creation of the composite, hydrogel, or nanofibers. Pore size can be from below about 1 nm to up to 100 nm, including 1, 2, 3, 4, 5, 10, 15, 20, 30, 40, 50, 60 70, 80, 90 or 100 nm, and the size thereof may be narrowly tailored, e.g., such that at least 40%, such as 50%, 60%, 70%, 80%, 90%, 95% or greater than 95% of the pores are in a desired size or within a desired size range.

The composite may be suitable for incorporation into a tissue of a human subject, and thus they are generally “biocompatible”, meaning capable of interacting with a biological system (such as found in a human subject) without inducing a pathophysiological response therein and/or thereby. In some embodiments, the composite is provided in order to be durably retained in the tissue, e.g., nerve tissues. Alternatively, the composite may be transiently retained in the human subject and are provided as substantially biodegradable. Preferably, the polymeric fibers or nanofibers in the composite include biocompatible biodegradable polymers, e.g., biocompatible biodegradable polyester. In certain embodiments, the polymeric fibers or nanofibers include polycaprolactone. In certain embodiments, the polymeric fibers or nanofibers are polycaprolactone.

To achieve fiber-reinforcement effect while maintaining high porosity in the hydrogel phase, an electrospun fiber-hydrogel composite that offers superior properties as compared to other complex is provided. Such a composite design not only allows stronger mechanical reinforcement from the solid fiber component, but also allows independent tuning of bulk mechanical properties and the average pore size/porosity of the hydrogel phase, enabling both optimal cell infiltration properties and structural integrity.

To further achieve the desired effects, in some embodiments, a PEG crosslinking agent is preferably used to introduce crosslinking between the nanofibers and also between the nanofibers and the hydrogel. This helps to extend durability of the product, and allows for modulation of crosslinking density in order to achieve optimal other properties.

### Gel/Hydrogel Component

In an aspect, the composite includes a hydrogel having three-dimensional network of polymers (e.g., hydrophilic polymers, hydrophobic polymers, blends thereof) held together by covalent or non-covalent crosslinks that can absorb a substantial amount of water (e.g., 50%, 60% 70%, 80%, 90%, 95%, 96%, 97%, 98%, 99% or greater than 99% per unit of non-water molecule) to form an elastic gel. In some embodiments, the hydrogel may be biodegradable. The hydrogel can include any type of suitable hydrogel component known in the art. The gel and/or hydrogels can be formed of any suitable synthetic or naturally-occurring materials.

In some embodiments, hydrogel materials are functionalized. In particular embodiments, hydrogel materials are functionalized with groups comprising hydroxyl, amino, carboxyl, thio, acrylate, sulfonate, phosphate, amide, as well as modified forms thereof, such as activated or protected forms.

The hyaluronic acid (HA) is preferably used as the hydrogel material. HA is a non-sulfated, linear polysaccharide with repeating disaccharide units which form the hydrogel component. HA is also a non-immunogenic, native component of the extracellular matrix in human tissues, and widely used as a dermal filler in aesthetic and reconstructive procedures.

Breakdown of HA is facilitated by native hyaluronidases whose expression is increased in areas of tissue damage and inflammation. Importantly, studies have shown that small HA degradation fragments of 3-10 disaccharide units are potent regulators of endothelial cell proliferation, migration, tubule formation, and angiogenesis. These biological functions of HA are thought to be mediated via CD44 in a pathway involving Ras and PKC. Blockade of CD44/HA interactions using anti-CD44 antibodies reduced proliferation and migration of human microvascular endothelial cells in vitro. HA hydrogels have been investigated as potential matrices for cell delivery in a variety of models of cell and tissue injury. These hydrogels can serve as a protective and supporting scaffold for cells and can also reduce scarring. Thus, it is believed HA has a critical role in enhancing tissue regeneration by promoting cell infiltration and promoting angiogenesis.

The molecular weight of hyaluronic acid may affect the overall properties of the composite. In some embodiments, the molecular wight of HA (e.g., HA-SH) may be at least about or greater than 10 kDa, at least about or greater than 50 kDa, at least about or greater than 100 kDa, at least about or greater than 200 kDa, at least about or greater than 300 kDa, at least about or greater than 400 kDa, at least about or greater than 500 kDa, at least about or greater than 600 kDa, at least about or greater than 700 kDa, at least about or greater than 800 kDa, at least about or greater than 900 kDa, at least about or greater than 1.0 MDa, at least about or greater than 1.5 MDa, at least about or greater than 2.0 MDa, at least about or greater than 2.5 MDa, at least about or greater than 3.0 MDa.

In some embodiments, hyaluronic acid are functionalized. In particular embodiments, hyaluronic acid are functionalized with groups comprising hydroxyl, amino, carboxyl, thio, acrylate, sulfonate, phosphate, amide, as well as modified forms thereof, such as activated or protected forms. In certain embodiments, the hydrogel material includes a hyaluronic acid (HA). In certain embodiments, the hydrogel material includes functionalized hyaluronic acid (HA). In other preferred embodiments, the hydrogel material includes acrylated hyaluronic acid (HA). In some embodiments, the hydrogel material includes thiolated hyaluronic acid (HA).

In some embodiment, the HA of the invention is a sterilized HA, e.g., chemically and/or physically sterilized.

Further, the polymer component of the hydrogels may also include a cellulose ester, for example, cellulose acetate, cellulose acetate propionate (CAP), cellulose acetate butyrate (CAB), cellulose propionate (CP), cellulose butyrate (CB), cellulose propionate butyrate (CPB), cellulose diacetate (CDA), cellulose triacetate (CTA), or the like. In some embodiments, the gels/hydrogels may include other water-swellable polymers, such as acrylate polymers, which are generally formed from acrylic acid, methacrylic acid, methyl acrylate, ethyl acrylate, methyl methacrylate, ethyl methacrylate, and/or other vinyl monomers.

### Nanofibers

In an aspect, the composite also includes polymeric fibers, generally having a mean diameter of from about 10 nm to about 10,000 nm, such as about 100 nm to about 8000 nm, or about 150 nm to about 5,000 nm, or about 100, 150, 200, 250, 300, 350, 400, 450, 500, 600, 700, 800, 900, 1,000, 1,500, 2,000, 2,500, 3,000, 3,500, 4,000, 4,500, 5,000, 5,500, 6,000, 6,500, 7,000, 7,500, or 8,000. The polymeric fiber generally has a mean length of from about 10 μm to about 500 μm, such as about 10, 50, 100, 150, 200, 250, 300, 350, 400, 450, or 500 μm.

In certain embodiments, the polymeric fibers are nanofibers generally having a mean diameter of less than about 999 nm, less than about 900 nm, less than about 800 nm, less than about 700 nm, less than about 600 nm, less than about 500 nm, less than about 400 nm, less than about 300 nm, less than about 200 nm, less than about 100 nm, less than about 50 nm, or less than about 10 nm. In some embodiments, the polymeric fibers generally have a length less than about 200 μm, less than about 250 μm, less than about 80 μm, less than about 50 μm, less than about 40 μm, less than about 30 μm, less than about 20 μm, or less than about 10 μm. The diameter and length of the nanofibers is determined using optical fluorescence microscopy or electron microscopy.

In some embodiments, nanofibers are functionalized. In some embodiments, fibers are functionalized with groups comprising hydroxyl, amino, carboxyl, thio, acrylate, sulfonate, phosphate, maleimide, amide, as well as modified forms thereof, such as activated or protected forms.

Particularly, the fibers (e.g., nanofibers or microfibers) in the fiber-hydrogel composite include one or more extracellular matrix proteins (ECMs). In certain embodiments, the fibers (e.g., nanofibers or microfibers) suitably include one or more selected from collagen, gelatin, elastin, elastin-like polypeptides, tropoelastin, decellularized matrix, and hyaluronic acid. In certain embodiments, the fibers (e.g., nanofibers or microfibers) include one or more from bovine type I collagen, gelatin, or derivatives.

Preferably, the polymeric fibers or nanofibers in the composite include biocompatible biodegradable polymers, e.g., biocompatible biodegradable polyester. In certain embodiments, the polymeric fibers or nanofibers include polycaprolactone. In certain embodiments, the polymeric fibers or nanofibers are polycaprolactone.

In certain embodiments, the fiber-hydrogel composite may include a natural extracellular matrix for fibers (e.g., nanofibers or microfibers). In certain embodiments, the fiber-hydrogel composite may include a synthetic extracellular matrix for fibers (e.g., nanofibers or microfibers). In certain embodiments, the one or more ECMs include a collagen nanofiber, which may be naturally obtained or synthesized. In certain embodiments, the collagen nanofiber includes a type I bovine collagen nanofiber or fragments thereof. In certain embodiments, the collagen nanofibers may be obtained from natural sources, or may be fabricated or prepared from a composition (resin composition) including collagen. For example, the collagen nanofiber may be formed by electrospinning, centrifugal spinning, blow spinning, or combinations thereof. Particularly, collagen nanofibers are preferably prepared by electrospinning.

In certain embodiments, microfabrication methods are used to make the nanofibers. In various embodiments, the disclosed devices can be assembled and/or manufactured using any suitable microfabrication technique. Such methods and techniques are widely known in the art.

The nanofibers may also be fabricated by electrostatic spinning (also referred to as electrospinning). The process of electrospinning generally involves the introduction of a liquid into an electric field, so that the liquid is caused to produce fibers. These fibers are generally drawn to a conductor at an attractive electrical potential for collection. During the conversion of the liquid into fibers, the fibers harden and/or dry. This hardening and/or drying may be caused by cooling of the liquid, i.e., where the liquid is normally a solid at room temperature; by evaporation of a solvent, e.g., by dehydration (physically induced hardening); or by a curing mechanism (chemically induced hardening).

Electrostatically spun fibers can be produced having very thin diameters. Parameters that influence the diameter, consistency, and uniformity of the electrospun fibers include the polymeric material and cross-linker concentration (loading) in the fiber-forming combination, the solvent composition, the applied voltage, and needle collector distance.

The electrospun fibers (e.g., collagen nanofiber) may provide superior properties, e.g., high porosity in the hydrogel phase and mechanical reinforcement from the solid fiber component, which may be beneficial for optimal cell infiltration properties and structural integrity.

In certain embodiments, collagen fibers are prepared by electrospinning the fibers in a solution or suspension containing 1,1,1,3,3,3-hexafluoro-2-propanol (HFIP or HFP) solvent. In other embodiments, the collagen fibers are prepared by electrospinning in a solution or suspension containing different solvents, such as trifluoroethanol (TFE), trifluoroacetic acid (TFA), acetic acid, ethanol, or phosphate mixtures. In certain embodiments, the suitable solvent may include at least one of 1,1,1,3,3,3 hexafluoro-2-propanol (HFIP), 2,2,2-trifluoroethanol (TFE), and a mixture of water and acetic acid. Other solvents that may be used or combined with other solvents in electrospinning natural matrix materials, such as collagen fibers, include acetamide, N-methylformamide, N,N-dimethylformamide (DMF), dimethylsulfoxide (DMSO), dimethylacetamide, N-methyl pyrrolidone (NMP), ethyl acetate, acetonitrile, trifluoroacetic anhydride, 1,1,1-trifluoroacetone, maleic acid, and hexafluoroacetone.

Selection of a solvent will depend upon the characteristics of the synthetic polymer to be electrodeposited, such as the secondary forces that stabilize polymer-polymer interactions and the solvent's ability to replace these with strong polymer-solvent interactions. In the case of polypeptides such as collagen, and in the absence of covalent cross-linking, the principal secondary forces between chains are: (1) Coulombic, resulting from attraction of fixed charges on the backbone and dictated by the primary structure (e.g., lysine and arginine residues will be positively charged at physiological pH, while aspartic or glutamic acid residues will be negatively charged); (2) dipole-dipole, resulting from interactions of permanent dipoles—the hydrogen bond, commonly found in polypeptides, is the strongest of such interactions; and (3) hydrophobic interactions, resulting from association of non-polar regions of the polypeptide due to a low tendency of non-polar species to interact favorably with polar water molecules.

Thus, solvents or solvent combinations that may favorably compete for these interactions may dissolve or disperse polypeptides. For example, HFP and TFE possess a highly polar hydroxyl group adjacent to a very hydrophobic fluorinated region. While not wishing to be bound by theory, it is believed that the alcohol portion may hydrogen bond with peptides, and may also solvate charges on the backbone, thus reducing Coulombic interactions between molecules. Additionally, the hydrophobic portions of these solvents may interact with hydrophobic domains in polypeptides, helping to resist the tendency of the latter to aggregate via hydrophobic interactions. Solvents such as HFP and TFE, due to their lower overall polarities compared to water, may not compete well for intramolecular hydrogen bonds that stabilize secondary structures such as an alpha helix. Consequently, alpha helices in these solvents are believed to be stabilized by virtue of stronger intramolecular hydrogen bonds. The stabilization of polypeptide secondary structures in these solvents is believed to be desirable, especially in the cases of collagen and elastin, to preserve the proper formation of collagen fibrils during electrospinning. In some embodiments, solvents are selected based on their tendency to induce helical structure in electrospun protein fibers, thereby predisposing monomers of collagen or other proteins to undergo polymerization and form helical polymers that mimic the native collagen fibril. Examples of such solvents include halogenated alcohols, preferably fluorinated alcohols (HFP and TFE), hexafluoroacetone, chloroalcohols in conjugation with aqueous solutions of mineral acids and dimethylacetamide, preferably containing lithium chloride. HFP and TFE are more preferred. In some embodiments, water is added to the solvents.

In certain embodiments, the collagen nanofibers are prepared using an alternate fiber stabilizer, vapor phase glutaraldehyde. Treatment with glutaraldehyde results in crosslinking of collagen fibers, as the aldehyde groups of glutaraldehyde react with the free lysine or hydroxylysine groups on collagen fibers to form Schiff base structures. For example, a six-hour vapor phase glutaraldehyde treatment results in increased tensile strength, elasticity, stretchability, and stability of collagen fibers. In certain embodiments, collagen nanofibers are prepared using alternate collagen stabilizers or crosslinkers, such as D-ribose. As disclosed in U.S. Pat. No. 4,971,954, incorporated herein in its entirety, D-ribose can crosslink collagen fibers, resulting in a non-toxic and non-immunogenic matrix.

In certain embodiments, EDC and fibers (e.g., nanofibers or microfibers) may be cross-linked (e.g., via a crosslinking moiety or directly linked). For example, form of interaction of EDC and fibers (e.g., nanofibers or microfibers) may be effective to introduce bonding (e.g., covalent bonding) therebetween.

The nanofibers may include, but not limited to, nanofibers, nanotubes, nanofilaments, mesh sections, branched filaments or networks. The nanofibers may also comprise any suitable chemical functional groups to facilitate the covalent or noncovalent crosslinking between the nanofibers and the polymers of the hydrogels of the invention.

Preferably, the nanofiber has a diameter ranging from about 10 nm to about 10,000 nm. In some embodiments, the nanofiber has a diameter in a range of about 10 nm to about 1000 nm. Further, the nanofiber may have an aspect ratio in a range of at least about 10 to about at least 200. It will be appreciated that, because of the very small diameter of the fibers, the fibers have a high surface area per unit of mass. This high surface area to mass ratio permits fiber-forming solutions or liquids to be transformed from liquid or solvated fiber-forming materials to solid nanofibers in fractions of a second.

In certain embodiments, the HA may be covalently bonded to the fibers (e.g., nanofibers or microfibers). For example, HA may be covalently bonded to the type I bovine collagen nanofiber of fragments thereof. In certain embodiments, the crosslinking agent generates interfacial bonding between the collagen nanofiber and the HA. Due to bonding and interaction (e.g., covalent, non-covalent or ionic bonding), the collagen nanofiber may be retained inside or inner space of the fiber-hydrogel composite (e.g., inside of the composite network). In certain embodiments, the crosslinking agent may react with the hydroxyl groups of the HA and amino groups of the collagen nanofiber to form the composite network. For example, interfacial bonding between the collagen nanofiber and the HA may increase the composite stiffness even at relatively low fiber loading density.

Due to the interfacial bonding and the formation of the composite network, the fiber-hydrogel composite may have increased cell permeability and/or maintains storage modulus. In certain embodiments, a storage modulus of the fiber-hydrogel composite is at least about 10 Pa, at least about 20 Pa, at least about 30 Pa, at least about 40 Pa, at least about 50 Pa, at least about 60 Pa, at least about 70 Pa, at least about 80 Pa, at least about 90 Pa, at least about 100 Pa, at least about 150 Pa, at least about 200 Pa, at least about 250 Pa, at least about 300 Pa, at least about 400 Pa, or at least about 500 Pa. In certain embodiments, a storage modulus of the fiber-hydrogel composite ranges from about 1 to about 1,000 Pa, from about 20 to about 800 Pa, from about 100 to about 500 Pa, or from about 150 to about 500 Pa. In certain embodiments, a storage modulus of the fiber-hydrogel composite ranges from about 0.5 to about 30 kPa.

In certain embodiments, an alternate hydrogel phase such as collagen, chitosan, alginate, PVA, gelatin, PEG, cellulose, or cellulose derivatives may be used in place of HA.

### Crosslinking

The preferred form of interaction of the complex/comp containing polymer fibers and hydrogel includes a crosslinking moiety, generally present in an amount effective to introduce bonding between polymer fiber and hydrogel material, e.g., to induce crosslinking between polycaprolactone fiber and hyaluronic acid.

For certain applications, particularly when high cohesive strength is desired, the polymers of the gel/hydrogels of the invention may be covalently crosslinked. The disclosure contemplates that crosslinking may be desired as between the polymers of the gel/hydrogel component, but also crosslinking may be desired as between the polymers of the gel/hydrogel and the nanostructure components of the composite materials of the invention. The invention contemplates any suitable means for crosslinking polymers to one another, and crosslinking the gel/hydrogel polymers with the nanostructure components of the invention. The gel/hydrogel polymers may be covalently crosslinked to other polymers or to the nanostructures, either intramolecularly or intermolecularly or through covalent bonds. In the former case, there are no covalent bonds linking the polymers to one another or to the nanostructures, while in the latter case, there are covalent crosslinks binding the polymers to one another or to the nanostructures. The crosslinks may be formed using any suitable means, including using heat, radiation, or a chemical curing (crosslinking) agent. The degree of crosslinking should be sufficient to eliminate or at least minimize cold flow under compression. Crosslinking also includes the use of a third molecule, a “cross-linker” utilized in the cross-linking process.

“Cross-linkers” or “Cross-linking agents” may be suitably chosen, for example, from the group of poly(ethylene glycol) PEG, e.g. thiolated poly(ethylene glycol), poly(ethylene glycol) diacrylate (PEGDA), or derivatives thereof. Examples of other cross-linking agents that may be suitable include DEO (diepoxyoctane), BDDE (1,4-butanediol diglycidyl ether), and DVS (divinyl sulfone).

For thermal crosslinking, a free radical polymerization initiator is used, and can be any of the known free radical-generating initiators conventionally used in vinyl polymerization. Preferred initiators are organic peroxides and azo compounds, generally used in an amount from about 0.01 wt. % to 15 wt. %, preferably 0.05 wt. % to 10 wt. %, more preferably from about 0.1 wt. % to about 5% and most preferably from about 0.5 wt. % to about 4 wt. % of the polymerizable material. Suitable organic peroxides include dialkyl peroxides such as t-butyl peroxide and 2,2bis(t-butylperoxy) propane, diacyl peroxides such as benzoyl peroxide and acetyl peroxide, peresters such as t-butyl perbenzoate and t-butyl per-2-ethylhexanoate, perdicarbonates such as dicetyl peroxy dicarbonate and dicyclohexyl peroxy dicarbonate, ketone peroxides such as cyclohexanone peroxide and methylethylketone peroxide, and hydroperoxides such as cumene hydroperoxide and tert-butyl hydroperoxide. Suitable azo compounds include azo bis (isobutyronitrile) and azo bis(2,4-dimethylvaleronitrile). The temperature for thermally crosslinking will depend on the actual components and may be readily deduced by one of ordinary skill in the art, but typically ranges from about 80° C. to about 200° C.

Crosslinking may also be accomplished with radiation, typically in the presence of a photoinitiator. The radiation may be ultraviolet, alpha, beta, gamma, electron beam, and x-ray radiation, although ultraviolet radiation is preferred. Useful photosensitizers are triplet sensitizers of the “hydrogen abstraction” type, and include benzophenone and substituted benzophenone and acetophenones such as benzyl dimethyl ketal, 4-acryloxybenzophenone (ABP), 1-hydroxy-cyclohexyl phenyl ketone, 2,2-diethoxyacetophenone and 2,2-dimethoxy-2-phenylaceto-phenone, substituted alpha-ketols such as 2-methyl-2-hydroxypropiophenone, benzoin ethers such as benzoin methyl ether and benzoin isopropyl ether, substituted benzoin ethers such as anisoin methyl ether, aromatic sulfonyl chlorides such as 2-naphthalene sulfonyl chloride, photoactive oximes such as 1-phenyl-1,2-propanedione-2-(O-ethoxy-carbonyl)-oxime, thioxanthones including alkyl- and halogen-substituted thioxanthonse such as 2-isopropylthioxanthone, 2-chlorothioxanthone, 2,4 dimethyl thioxanone, 2,4 dichlorothioxanone, and 2,4-diethyl thioxanone, and acyl phosphine oxides. Radiation having a wavelength of 200 to 800 nm, preferably, 200 to 500 nm, is preferred for use herein, and low intensity ultraviolet light is sufficient to induce crosslinking in most cases. However, with photosensitizers of the hydrogen abstraction type, higher intensity UV exposure may be necessary to achieve sufficient crosslinking. Such exposure can be provided by a mercury lamp processor such as those available from PPG, Fusion, Xenon, and others. Crosslinking may also be induced by irradiating with gamma radiation or an electron beam. Appropriate irradiation parameters, i.e., the type and dose of radiation used to effect crosslinking, will be apparent to those skilled in the art.

Suitable chemical curing agents, also referred to as chemical cross-linking “promoters,” include, without limitation, polymercaptans such as 2,2-dimercapto diethylether, dipentaerythritol hexa (3-mercaptopropionate), ethylene bis(3-mercaptoacetate), pentaerythritol tetra(3-mercaptopropionate), pentaerythritol tetrathioglycolate, polyethylene glycol dimercaptoacetate, polyethylene glycol di(3-mercaptopropionate), trimethylolethane tri (3-mercaptopropionate), trimethylolethane trithioglycolate, trimethylolpropane tri (3-mercaptopropionate), trimethylolpropane trithioglycolate, dithioethane, di- or trithiopropane and 1,6-hexane dithiol. The crosslinking promoter is added to the uncrosslinked hydrophilic polymer to promote covalent crosslinking thereof, or to a blend of the uncrosslinked hydrophilic polymer and the complementary oligomer, to provide crosslinking between the two components.

The polymers and/or nanostructures may also be crosslinked prior to admixture with the complementary oligomer. In such a case, it may be preferred to synthesize the polymer in crosslinked form, by admixing a monomeric precursor to the polymer with multifunctional comonomer and copolymerizing. Polymerization may be carried out in bulk, in suspension, in solution, or in an emulsion. Solution polymerization is preferred, and polar organic solvents such as ethyl acetate and lower alkanols (e.g., ethanol, isopropyl alcohol, etc.) are particularly preferred.

In some embodiments, is a chemical crosslinking agent is employed, the amount used will preferably be such that the weight ratio of crosslinking agent to hydrophilic polymer is in the range of about 1:100 to 1:5. To achieve a higher crosslink density, if desired, chemical crosslinking is combined with radiation curing.

In some embodiments, the crosslinking agent includes poly(ethylene glycol) diacrylate (PEGDA), or a derivative thereof. In certain embodiments, when the functionalized hyaluronic acid comprises thiolated hyaluronic acid, the crosslinking agent includes poly(ethylene glycol) diacrylate (PEGDA), or a derivative thereof.

### Microbeads

As discussed, in various aspects, it can be preferred that the composites/hydrogels are formed into particulate formulations, enabling use of higher concentrations of each component and enhanced stability. In a preferred aspect, a system of particulation may be employed wherein the pre-formed hydrogel-nanofiber composite is physically modulated, such as by being pushed through one, two, three, or more than three mesh screens, creating a population of nonspherical beads that are relatively similar to one another in shape and size. This two-screen system allows for tight control over the size of the beads, thus allowing the user to modulate the size as needed. Such non-spherical microbeads are disclosed in US 2020/0069846 and U.S. Pat. No. 11,771,807.

### Active Agents

Any of the herein-described gel/hydrogel compositions may be utilized so as to contain an active agent and thereby act as an active agent delivery system when applied to a body surface (e.g., a site of tissue repair) in active agent-transmitting relation thereto. The release of active agents “loaded” into the hydrogel or composite typically involves both absorption of water and desorption of the agent via a swelling-controlled diffusion mechanism. For example, active agent-containing hydrogel compositions may be employed, by way of example, in transdermal drug delivery systems, in wound dressings, in topical pharmaceutical formulations, in implanted drug delivery systems, in oral dosage forms, and the like.

Suitable active agents that may be incorporated into the present hydrogel compositions and delivered systemically (e.g., with a transdermal, oral, or other dosage form suitable for systemic administration of a drug) include, but are not limited to: analeptic agents; analgesic agents; anesthetic agents; antiarthritic agents; respiratory drugs, including antiasthmatic agents; anticancer agents, including antineoplastic drugs; anticholinergics; anticonvulsants; antidepressants; antidiabetic agents; antidiarrheals; antihelminthics; antihistamines; antihyperlipidemic agents; antihypertensive agents; anti-infective agents such as antibiotics and antiviral agents; antiinflammatory agents; antimigraine preparations; antinauseants; antiparkinsonism drugs; antipruritics; antipsychotics; antipyretics; antispasmodics; antitubercular agents; antiulcer agents; antiviral agents; anxiolytics; appetite suppressants; attention deficit disorder (ADD) and attention deficit hyperactivity disorder (ADHD) drugs; cardiovascular preparations including calcium channel blockers, antianginal agents, central nervous system (CNS) agents, beta-blockers and antiarrhythmic agents; central nervous system stimulants; cough and cold preparations, including decongestants; diuretics; genetic materials; herbal remedies; hormonolytics; hypnotics; hypoglycemic agents; immunosuppressive agents; leukotriene inhibitors; mitotic inhibitors; muscle relaxants; narcotic antagonists; nicotine; nutritional agents, such as vitamins, essential amino acids and fatty acids; ophthalmic drugs such as antiglaucoma agents; parasympatholytics; peptide drugs; psychostimulants; sedatives; steroids, including progestogens, estrogens, corticosteroids, androgens and anabolic agents; smoking cessation agents; sympathomimetics; tranquilizers; and vasodilators including general coronary, peripheral and cerebral. Specific active agents with which the present adhesive compositions are useful include, without limitation, anabasine, capsaicin, isosorbide dinitrate, aminostigmine, nitroglycerine, verapamil, propranolol, silabolin, foridone, clonidine, cytisine, phenazepam, nifedipine, fluacizin, and salbutamol.

For topical drug administration and/or medicated cushions (e.g., medicated footpads), suitable active agents include, by way of example, the following:
Bacteriostatic and bactericidal agents: Suitable bacteriostatic and bactericidal agents include, by way of example: halogen compounds such as iodine, iodopovidone complexes (i.e., complexes of PVP and iodine, also referred to as “povidine” and available under the tradename Betadine from Purdue Frederick), iodide salts, chloramine, chlorohexidine, and sodium hypochlorite; silver and silver-containing compounds such as sulfadiazine, silver protein acetyltannate, silver nitrate, silver acetate, silver lactate, silver sulfate and silver chloride; organotin compounds such as tri-n-butyltin benzoate; zinc and zinc salts; oxidants, such as hydrogen peroxide and potassium permanganate; aryl mercury compounds, such as phenylmercury borate or merbromin; alkyl mercury compounds, such as thiomersal; phenols, such as thymol, o-phenyl phenol, 2-benzyl-4-chlorophenol, hexachlorophen and hexylresorcinol; and organic nitrogen compounds such as 8-hydroxyquinoline, chlorquinaldol, clioquinol, ethacridine, hexetidine, chlorhexedine, and ambazone.

Antibiotic agents: Suitable antibiotic agents include, but are not limited to, antibiotics of the lincomycin family (referring to a class of antibiotic agents originally recovered from streptomyces lincolnensis), antibiotics of the tetracycline family (referring to a class of antibiotic agents originally recovered from streptomyces aureofaciens), and sulfur-based antibiotics, i.e., sulfonamides. Exemplary antibiotics of the lincomycin family include lincomycin, clindamycin, related compounds, and pharmacologically acceptable salts and esters thereof. Exemplary antibiotics of the tetracycline family include tetracycline itself, chlortetracycline, oxytetracycline, tetracycline, demeclocycline, rolitetracycline, methacycline and doxycycline and their pharmaceutically acceptable salts and esters, particularly acid addition salts such as the hydrochloride salt. Exemplary sulfur-based antibiotics include, but are not limited to, the sulfonamides sulfacetamide, sulfabenzamide, sulfadiazine, sulfadoxine, sulfamerazine, sulfamethazine, sulfamethizole, sulfamethoxazole, and pharmacologically acceptable salts and esters thereof, e.g., sulfacetamide sodium.

Pain relieving agents: Suitable pain relieving agents are local anesthetics, including, but not limited to, acetamidoeugenol, alfadolone acetate, alfaxalone, amucaine, amolanone, amylocaine, benoxinate, betoxycaine, biphenamine, bupivacaine, burethamine, butacaine, butaben, butanilicaine, buthalital, butoxycaine, carticaine, 2-chloroprocaine, cinchocaine, cocaethylene, cocaine, cyclomethycaine, dibucaine, dimethisoquin, dimethocaine, diperadon, dyclonine, ecgonidine, ecgonine, ethyl aminobenzoate, ethyl chloride, etidocaine, etoxadrol, .beta.-eucaine, euprocin, fenalcomine, fomocaine, hexobarbital, hexylcaine, hydroxydione, hydroxyprocaine, hydroxytetracaine, isobutyl p-aminobenzoate, kentamine, leucinocaine mesylate, levoxadrol, lidocaine, mepivacaine, meprylcaine, metabutoxycaine, methohexital, methyl chloride, midazolam, myrtecaine, naepaine, octacaine, orthocaine, oxethazaine, parethoxycaine, phenacaine, phencyclidine, phenol, piperocaine, piridocaine, polidocanol, pramoxine, prilocaine, procaine, propanidid, propanocaine, proparacaine, propipocaine, propofol, propoxycaine, pseudococaine, pyrrocaine, risocaine, salicyl alcohol, tetracaine, thialbarbital, thimylal, thiobutabarbital, thiopental, tolycaine, trimecaine, zolamine, and combinations thereof. Tetracaine, lidocaine and prilocaine are referred pain relieving agents herein.

Other topical agents that may be delivered using the present hydrogel compositions as drug delivery systems include the following: antifungal agents such as undecylenic acid, tolnaftate, miconazole, griseofulvine, ketoconazole, ciclopirox, clotrimazole and chloroxylenol; keratolytic agents, such as salicylic acid, lactic acid and urea; vessicants such as cantharidin; anti-acne agents such as organic peroxides (e.g., benzoyl peroxide), retinoids (e.g., retinoic acid, adapalene, and tazarotene), sulfonamides (e.g., sodium sulfacetamide), resorcinol, corticosteroids (e.g., triamcinolone), alpha-hydroxy acids (e.g., lactic acid and glycolic acid), alpha-keto acids (e.g., glyoxylic acid), and antibacterial agents specifically indicated for the treatment of acne, including azelaic acid, clindamycin, erythromycin, meclocycline, minocycline, nadifloxacin, cephalexin, doxycycline, and ofloxacin; skin-lightening and bleaching agents, such as hydroquinone, kojic acid, glycolic acid and other alpha-hydroxy acids, artocarpin, and certain organic peroxides; agents for treating warts, including salicylic acid, imiquimod, dinitrochlorobenzene, dibutyl squaric acid, podophyllin, podophyllotoxin, cantharidin, trichloroacetic acid, bleomycin, cidofovir, adefovir, and analogs thereof, and anti-inflammatory agents such as corticosteroids and nonsteroidal anti-inflammatory drugs (NSAIDs), where the NSAIDS include ketoprofen, flurbiprofen, ibuprofen, naproxen, fenoprofen, benoxaprofen, indoprofen, pirprofen, carprofen, oxaprozin, pranoprofen, suprofen, alminoprofen, butibufen, fenbufen, and tiaprofenic acid.

For wound dressings, suitable active agents are those useful for the treatment of wounds, and include, but are not limited to bacteriostatic and bactericidal compounds, antibiotic agents, pain relieving agents, vasodilators, tissue-healing enhancing agents, amino acids, proteins, proteolytic enzymes, cytokines, and polypeptide growth factors.

For topical and transdermal administration of some active agents, and in wound dressings, it may be necessary or desirable to incorporate a permeation enhancer into the hydrogel composition in order to enhance the rate of penetration of the agent into or through the skin. Suitable enhancers include, for example, the following: sulfoxides such as dimethylsulfoxide (DMSO) and decylmethylsulfoxide; ethers such as diethylene glycol monoethyl ether (available commercially as Transcutol) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80) and lecithin (U.S. Pat. No. 4,783,450); the 1-substituted azacycloheptan-2-ones, particularly 1-n-dodecylcyclaza-cycloheptan-2-one (available under the trademark Azone from Nelson Research & Development Co., Irvine, Calif.; see U.S. Pat. Nos. 3,989,816, 4,316,893, 4,405,616 and 4,557,934); alcohols such as ethanol, propanol, octanol, decanol, benzyl alcohol, and the like; fatty acids such as lauric acid, oleic acid and valeric acid; fatty acid esters such as isopropyl myristate, isopropyl palmitate, methylpropionate, and ethyl oleate; polyols and esters thereof such as propylene glycol, ethylene glycol, glycerol, butanediol, polyethylene glycol, and polyethylene glycol monolaurate (PEGML; see, e.g., U.S. Pat. No. 4,568,343); amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine; terpenes; alkanones; and organic acids, particularly salicylic acid and salicylates, citric acid and succinic acid. Mixtures of two or more enhancers may also be used.

In certain other embodiments, the composite compositions including hydrogel component and nanofibers may also comprise additional optional additive components. Such components are known in the art and can include, for example, fillers, preservatives, pH regulators, softeners, thickeners, pigments, dyes, refractive particles, stabilizers, toughening agents, detackifiers, pharmaceutical agents (e.g., antibiotics, angiogenesis promoters, antifungal agents, immunosuppressing agents, antibodies, and the like), and permeation enhancers. These additives, and amounts thereof, are selected in such a way that they do not significantly interfere with the desired chemical and physical properties of the hydrogel composition.

Absorbent fillers may be advantageously incorporated to control the degree of hydration when the adhesive is on the skin or other body surface. Such fillers can include microcrystalline cellulose, talc, lactose, kaolin, mannitol, colloidal silica, alumina, zinc oxide, titanium oxide, magnesium silicate, magnesium aluminum silicate, hydrophobic starch, calcium sulfate, calcium stearate, calcium phosphate, calcium phosphate dihydrate, woven and non-woven paper and cotton materials. Other suitable fillers are inert, i.e., substantially non-adsorbent, and include, for example, polyethylenes, polypropylenes, polyurethane polyether amide copolymers, polyesters and polyester copolymers, nylon and rayon.

The compositions can also include one or more preservatives. Preservatives include, by way of example, p-chloro-m-cresol, phenylethyl alcohol, phenoxyethyl alcohol, chlorobutanol, 4-hydroxybenzoic acid methylester, 4-hydroxybenzoic acid propylester, benzalkonium chloride, cetylpyridinium chloride, chlorohexidine diacetate or gluconate, ethanol, and propylene glycol.

The compositions may also include pH regulating compounds. Compounds useful as pH regulators include, but are not limited to, glycerol buffers, citrate buffers, borate buffers, phosphate buffers, or citric acid-phosphate buffers may also be included so as to ensure that the pH of the hydrogel composition is compatible with that of an individual's body surface.

The compositions may also include suitable softening agents. Suitable softeners include citric acid esters, such as triethylcitrate or acetyl triethylcitrate, tartaric acid esters such as dibutyltartrate, glycerol esters such as glycerol diacetate and glycerol triacetate; phthalic acid esters, such as dibutyl phthalate and diethyl phthalate; and/or hydrophilic surfactants, preferably hydrophilic non-ionic surfactants, such as, for example, partial fatty acid esters of sugars, polyethylene glycol fatty acid esters, polyethylene glycol fatty alcohol ethers, and polyethylene glycol sorbitan-fatty acid esters.

The compositions may also include thickening agents. Preferred thickeners herein are naturally occurring compounds or derivatives thereof, and include, by way of example: collagen; galactomannans; starches; starch derivatives and hydrolysates; cellulose derivatives such as methyl cellulose, hydroxypropylcellulose, hydroxyethyl cellulose, and hydroxypropyl methyl cellulose; colloidal silicic acids; and sugars such as lactose, saccharose, fructose and glucose. Synthetic thickeners such as polyvinyl alcohol, vinylpyrrolidone-vinylacetate-copolymers, polyethylene glycols, and polypropylene glycols may also be used.

In certain embodiments, the hydrogel composite of the invention comprising a hydrogel and nanofibers further comprises a component that promotes angiogenesis. A challenge to achieving clinically relevant soft tissue regeneration prior to the present invention is that the regenerated tissue preferably should be re-vascularized. Therefore, any material that promotes soft tissue regeneration preferably should also encourage angiogenesis. One way to achieve this is through the use of heparin-containing hydrogel components, which can serve as growth factor binding sites to enrich and retain growth factors promoting angiogenesis and tissue formation.

In an embodiment, the composition further comprises and delivers an antibody. The term “antibody” is used herein in its broadest sense and includes certain types of immunoglobulin molecules comprising one or more antigen-binding domains that specifically bind to an antigen or epitope. An antibody specifically includes intact antibodies (e.g., intact immunoglobulins), antibody fragments, and multi-specific antibodies.

In some embodiments, the antibody comprises an antibody. In some aspects, the antibody is a monoclonal antibody. In some aspects, the antibody is a chimeric antibody. In some aspects, the antibody is a humanized antibody. In some aspects, the antibody is a human antibody. In some aspects, the antibody comprises an antibody fragment. In some embodiments, the antibody comprises an alternative scaffold.

In an embodiment, the compositions provided herein further comprise cells for delivery. In some embodiments, the cells are derived from the subject to whom they are administered. In some aspects, the cells are derived from a source other than the subject to whom they are administered. In some aspects, the cells are derived from a cell line. In some aspects, the cells are derived from a human source. In some aspects, the cells are derived from a humanized animal source.

In some aspects, the cells provided are stem cells. In some aspects, the cells provided are nerve cells.

In some embodiments, compositions provided herein further comprise small molecules for delivery, wherein the small molecule is a biologically active material. In some embodiments, the small molecule can cause pharmacological activity or anther direct effect in the diagnosis, cure, mitigation, treatment or prevention of disease or can affect the structure or function of the body.

The gel/hydrogel/nanofiber composites of the invention can also include tissue-repairing agents, such as, a number of growth factors, including epidermal growth factor (EDF), PDGF, and nerve growth factors (NGF's). For example, the compositions may include EGF. Epidermal Growth Factor (EGF) was discovered after the observation that cutaneous wounds in laboratory mice seemed to heal more rapidly when the mice were allowed to lick them. This was not simply due to some antiseptic agent in saliva (such as lysozyme). A specific growth factor, now known as EGF, was shown to be responsible. EGF is identical to urogastrone and has angiogenic properties. Transforming growth factor-alpha (TGFα) is very similar, binding to the same receptor and is even more effective in stimulating epithelial cell regeneration (epithelisation).

Thus, hydrogels including EGF/TGF may advantageously be used in the acceleration of wound healing and burns, reduction in keloid scar formation (especially for burns), skin engraftment dressings, and the treatment of chronic leg ulcers.

Tissue-repairing agents useful in the present invention include a number of growth factors, including epidermal growth factor (EDF), PDGF, and nerve growth factors (NGF's). Generally, growth-promoting hormones will affect between one and four tissues. Many of the products developed from such proteins are targeted towards wound repairs of one kind or another, although there are other indications. Some of the most important tissue growth factors are described further below.

The gel/nanofibers compositions of the invention may also include one or more growth factors that may be useful in the tissue repair methods and other applications of the invention.

The hydrogel/nanofibers compositions of the invention may also include VEGF to promote angiogenesis. Vascular Endothelial Growth Factor (VEGF—also known as vascular permeability factor) is another vascular growth factor that is a multifunctional angiogenic cytokine. It contributes to angiogenesis (blood vessel growth) both indirectly and directly by stimulating proliferation of endothelial cells at the microvessel level, causing them to migrate and to alter their generic expression. VEGF also makes theses endothelial cells hyperpermeable, causing them to release plasma proteins outside the vascular space, which causes changes in the area, contributing to angiogenesis.

The compositions of the invention may also include FGF. Fibroblast Growth Factor (FGF) is actually a family of at least 19 different 14-18 kD peptides belonging to the heparin-binding growth factors family and are mitogenic for cultured fibroblasts and vascular endothelial cells. They are also angiogenic in vivo and this angiogenicity is enhanced by TNF. FGF's may be used in a similar manner to EGF. bFGF, also known as FGF-2, is involved in controlling human megakaryocytopoiesis and FGFs have been shown to be effective in stimulating endothelial cell formation, and in assisting in connective tissue repair.

Hydrogel/nanofibers compositions may also comprise Keratinocyte Growth Factor (KGF), also known as FGF-7, for use in wound healing and other disorders involving epithelial cell destruction.

Transforming Growth Factors (TGF's) have the ability to transform various cell lines, and can confer, for example, the ability to grow in culture for more than a limited number of generations, growth in multiple layers rather than monolayers, and the acquisition of an abnormal karyotype. There are at least five members of the TGF family, the two most widely studied being TGF-alpha and TGF-beta. The former is mitogenic for fibroblasts and endothelial cells, angiogenic, and promotes bone resorption. Compositions also may include TGF. TGF-beta is a general mediator of cell regulation, a powerful inhibitor of cell growth, and inhibits the proliferation of many cell types. TGF-beta can antagonize the mitogenic effects of other peptide growth factors and can also inhibit the growth of many tumour cell lines. TGF-beta also has angiogenic effects and promotes collagen formation in fibroblasts.

Hydrogel/nanofiber compositions of the present invention may usefully comprise free, uncrosslinked collagen, for example. Although collagen, in this form, is unlikely to serve a useful structural function, it primarily serves as a sacrificial protein where proteolytic activity is undesirably high, thereby helping to prevent maceration of healthy tissue, for example.

Hydrogel/nanofiber compositions can also include certain enzymes. Enzymes are used in the debridement of both acute and chronic wounds. Debridement is the removal of nonviable tissue and foreign matter from a wound and is a naturally occurring event in the wound-repair process. During the inflammatory phase, neutrophils and macrophages digest and remove “used” platelets, cellular debris, and avascular injured tissue from the wound area. However, with the accumulation of significant amounts of damaged tissue, this natural process becomes overwhelmed and insufficient. Build-up of necrotic tissue then places considerable phagocytic demand on the wound and retards wound healing. Consequently, debridement of necrotic tissue is a particular objective of topical therapy and an important component of optimal wound management.

Enzymes, for example, may be incorporated into hydrogels of the present invention for topical application to provide a selective method of debridement. Suitable enzymes may be derived from various sources, such as krill, crab, papaya, bovine extract, and bacteria Commercially available, suitable enzymes include collagenase, papain/urea, and a fibrinolysin and deoxyribonuclease combination.

Enzymes for use in the present invention generally work in one of two ways: by directly digesting the components of slough (e.g., fibrin, bacteria, leukocytes, cell debris, serous exudate, DNA); or, by dissolving the collagen “anchors” that secure the avascular tissue to the underlying wound bed.

Hydrogels of the present invention may comprise Dakin's solution, if desired, generally to exert antimicrobial effects and odor control. As a debridement agent, Dakin's solution is non-selective because of its cytotoxic properties. Dakin's solution denatures protein, rendering it more easily removed from the wound. Loosening of the slough also facilitates debridement by other methods. Hydrogels comprising Dakin's solution may be changed twice daily if the goal is debridement. Periwound skin protection should generally be provided with ointments, liquid skin barrier film dressings, or solid skin barrier wafers, for example.

The gel of the present invention may be delivered by any suitable method, such as via a syringe or bellows pack (single dose delivery systems) or a multidose system, such as a pressurized delivery system or delivery via a ‘bag in the can’ type system.

As such, the present invention also extends to a single dose delivery system comprising a gel according to the present invention, for the treatment of wounds. The invention also extends to a pressurized delivery system comprising a gel according to the present invention, and a pressurized hydrogel according to the present invention in an aerosol container capable of forming a spray upon release of pressure therefrom. Use of such delivery means allows the gel to be delivered to areas on a patient which are otherwise difficult to reach by direct application.

In certain embodiment, it may be advantageous to render the hydrogel compositions of the invention electrically conductive for use in biomedical electrodes and other electrotherapy contexts, i.e., to attach an electrode or other electrically conductive member to the body surface. For example, the hydrogel composition may be used to attach a transcutaneous nerve stimulation electrode, an electrosurgical return electrode, or an EKG electrode to a patient's skin or mucosal tissue. These applications involve modification of the hydrogel composition so as to contain a conductive species. Suitable conductive species are ionically conductive electrolytes, particularly those that are normally used in the manufacture of conductive adhesives used for application to the skin or other body surface, and include ionizable inorganic salts, organic compounds, or combinations of both. Examples of ionically conductive electrolytes include, but are not limited to, ammonium sulfate, ammonium acetate, monoethanolamine acetate, diethanolamine acetate, sodium lactate, sodium citrate, magnesium acetate, magnesium sulfate, sodium acetate, calcium chloride, magnesium chloride, calcium sulfate, lithium chloride, lithium perchlorate, sodium citrate and potassium chloride, and redox couples such as a mixture of ferric and ferrous salts such as sulfates and gluconates. Preferred salts are potassium chloride, sodium chloride, magnesium sulfate, and magnesium acetate, and potassium chloride is most preferred for EKG applications. Although virtually any amount of electrolyte may be present in the adhesive compositions of the invention, it is preferable that any electrolyte present be at a concentration in the range of about 0.1 to about 15 wt. % of the hydrogel composition. The procedure described in U.S. Pat. No. 5,846,558 to Nielsen et al. for fabricating biomedical electrodes may be adapted for use with the hydrogel compositions of the invention, and the disclosure of that patent is incorporated by reference with respect to manufacturing details. Other suitable fabrication procedures may be used as well, as will be appreciated by those skilled in the art.

### Non-Spherical Beaded Formulation

The composites/hydrogels may be formed into particulate formulations (e.g., beads, particles, or microbeads) enabling use of higher concentrations of each component, easing gel delivery, improving the mixing with biologically-active components, and enhancing stability. In the related art, some commercial hydrogel-based fillers may be blended with blades or similar in order to form beads. However, this method is not ideal because it allows for little control over bead size and shape. Further, the nanoparticle-hydrogel composite is formed as a bulk composite gel or solidified gel, which generally does not produce to a uniform solid material.

Provided are improvements including introducing the composite as a beaded gel. This allows for the user to vary the bead properties in order to get desirable results and improves the storage modulus of the composite. In certain embodiments, the pre-formed hydrogel-nanofiber composite is physically modulated to form particulates, such as by being pushed through one, two, three, or more than three mesh screens, creating a population of nonspherical beads that are relatively similar to one another in shape and size. This multi-screen system allows for tight control over the size of the beads, thus allowing the user to modulate the size as needed.

### Pre-Reacted Composite

Preferably, the complex or hydrogel composite may be reacted prior to injection or prior to storage. If the components of the formulation are mixed by the end-user immediately prior to injection into a subject, the application of the composite may include complications of human factors, e.g. the user preparation and handling that this necessitated, and mixing and wait times can change the reaction time and thus significantly alter the stiffness of the complex. For example, the gel may become too stiff to be injected through a syringe, or not stiff enough, which would create undesirable properties when injected into a subject. To address this problem, the inventors developed a pre-reacted composition, wherein the reaction (e.g gelation) takes place during manufacturing prior to storage.

In certain embodiments, the hydrogel composite is gelated or cured and milled or screened to form the beads, particles, or microbeads. In certain embodiments, after gelling, the hydrogel composite is made into beads by forcing the gel through a mesh or a screen. The bead sizes were varied by varying the mesh size of the screens used in the beading process. Preferably, the size of opening of the mesh or screen ranges from about 90 μm to about 250 μm, from about 95 μm to about 200 μm, or about 100 μm to about 150 μm.

Bead screen sets with the finest openings still greater than 250 μm may not be suitable for a typical injection setting, since the resulting beads need to have at least one dimension that is sized smaller than the inner diameter of the syringe needle. The needles commonly used for dermal filler applications range from 25-gauge to 30-gauge, with an inner diameter of from 260 μm to 160 μm. The practicable lower screen size may be limited by the size of fiber component, due to the risk of fiber-gel disruption if the individual fibers are larger in length than the screen openings. For this reason, meshes with openings larger than 20 microns are used.

### Lyophilization

The hydrogel composition, e.g., beads (e.g., microbeads) or particles, may be process by lyophilization prior to storage. The introduction of lyophilization allows for storage of the product at room temperature for extended periods of time without loss of function and allows flexibility for rehydrating fluid. Preferably, the beaded product is lyophilized in an isotonic solution of sucrose, Trehalose, and sodium chloride. These variables protect the microstructure during the drying process and extend the product's shelf life. The lyophilized gel beads (e.g., microbeads) may be reconstituted with water after storage, allowing them to be ready for injection within seconds.

The mechanical properties of the nanofiber phase of the fiber-hydrogel composite do not substantially change in the dried or frozen state, as opposed to most hydrogel components. Thus, during freezing or lyophilization, the fiber fraction can help maintain the overall composite microstructure. With the correct lyophilization cycle and formulation, the composite can be lyophilized, while still remaining as distinct beads (e.g., microbeads) upon rehydration.

### Soft Tissue Device

Provided herein is a soft tissue device or implant that includes a biologically active material, and non-spherical microbeads comprising a hydrogel composite. The hydrogel composite includes a functionalized hyaluronic acid network covalently linked to a plurality of polycaprolactone nanofibers as described herein.

Preferably, the mean size of the non-spherical microbeads is within the range of about 50 micrometers to about 300 micrometers along the longest dimension.

Preferably, the biologically active material may be suitably for fat grafting, e.g., which may be differentiated into soft tissues such as fat, when supported with a suitable matrix microenvironment. In certain embodiments, the biologically active material includes a population of adipose cells, autologous adipose cells, allogenic cells, genetically modified allogenic cells, stem cells, mesenchymal stem cells, genetically modified stem cells, genetically modified allogenic induced pluripotent stem (iPS) cells, genetically modified hypoimmunogenic pluripotent stem cells, adipose stromal vascular fraction, adipose tissue, autologous adipose tissue, lipoaspirate, a derivative thereof, or a combination thereof.

In some embodiments, the biologically active material includes adipose tissue. In some embodiments, the adipose tissue is lipoaspirate. In some embodiments, the adipose tissue is autologous.

In certain embodiments, the soft tissue device or implant may suitably include the non-spherical microbeads in a volume of about 25% to 75% of the total volume of the soft tissue device. In certain embodiments, the soft tissue device or implant may suitably include the non-spherical microbeads in a volume of about 30% to 70% of the total volume of the soft tissue device. In certain embodiments, the soft tissue device or implant may suitably include the non-spherical microbeads in a volume of about 35% to 65% of the total volume of the soft tissue device. In certain embodiments, the soft tissue device or implant may suitably include the non-spherical microbeads in a volume of about 40% to 60% of the total volume of the soft tissue device. In certain embodiments, the soft tissue device or implant may suitably include the non-spherical microbeads in a volume of about 50% of the total volume of the soft tissue device.

In certain embodiments, the soft tissue device or implant may suitably include the non-spherical microbeads in an amount of about 10% to 90% of the total weight of the soft tissue device. In certain embodiments, the soft tissue device or implant may suitably include the non-spherical microbeads in an amount of about 20% to 80% of the total weight of the soft tissue device. In certain embodiments, the soft tissue device or implant may suitably include the non-spherical microbeads in an amount of about 30% to 70% of the total weight of the soft tissue device. In certain embodiments, the soft tissue device or implant may suitably include the non-spherical microbeads in an amount of about 40% to 60% of the total weight of the soft tissue device.

In certain embodiments, the biological material is formed in fat particles. Preferably, a ratio of the mean size of the non-spherical microbeads to a mean size of the fat particles ranges from about 10:1 to about 1:10. In some embodiments, a ratio of the mean size of the non-spherical microbeads to a mean size of the fat particles is about 10:1, about 3:1, about 1:1, about 1:2, about 1:3, or about 1:10.

The fat particles may be prepared using non-limiting methods in any clinical use. For example, the fat particles may be processed by forming a fat film and digesting the film using mechanical forces, e.g., shear force, milling or grinding. In certain embodiments, the fat particles may be obtained from animal body fat (e.g., fish oil or mammalian fat) using aspirator or vacuum suction. The animal fat may be further processed by using physical forces such as harvesting, milling or grinding, to make homogenized fat particles or to obtain even distribution of the particle sizes. In certain embodiments, the size or diameter (e.g., mean diameter) of the far particle may be controlled using a filter, or filtration apparatus. In certain embodiments, the size or diameter (e.g., mean diameter) of the fat particle may be less than about 1,000 μm, less than about 900 μm, less than about 800 μm, less than about 700 μm, less than about 600 μm, less than about 500 μm, less than about 400 μm, less than about 300 μm, less than about 200 μm, less than about 100 μm, or preferably range from 10 to 90 μm, or particularly range from 20 to 80 μm.

In certain embodiments, the microbeads is gelated or cured before the biological active material is added to the microbeads. The microbeads may be gelated by chemical reaction (e.g., cross-linking) or by UV irradiation to cure or polymerize the polymers to form nanofibers.

In certain embodiments, the device or implant may include the hydrogel composition made from in thiolated hyaluronic acid and a crosslinking agent selected from poly(ethylene glycol) diacrylate (PEGDA), or a derivative thereof. In certain embodiments, the device or implant may include the hydrogel composition made from acrylated hyaluronic acid and a crosslinking agent selected from thiolated poly(ethylene glycol), or a derivative thereof.

In certain embodiments, the plurality of polycaprolactone fibers is made by electrospinning. The plurality of polycaprolactone fibers suitably includes an electrospun fiber.

In some embodiments, the soft tissue device or implant may further include a compound selected from the group consisting of growth factors, compounds stimulating angiogenesis, immunomodulators, inhibitors of inflammation, and combinations thereof. In some embodiments, the soft tissue device may further include one or more compounds that have therapeutic effects, vascularization effects, anti-vascularization effects, anti-inflammatory effects, anti-bacterial effects, antihistamine effects, and combinations thereof.

Preferably, the soft tissue device has a tan delta value of less than about 0.27. The tan delta is the rheological loss modulus divided by the storage modulus, which means that a lower tan delta number equates to a more “solid-like” as opposed to “liquid-like” material. The tan delta may also indicate a rheological property, which may vary based on the oil or fat contents of the material or substance.

In certain embodiments, the microbeads are substantially stable at room temperature for at least about 1 week, at least about 2 weeks, at least about 1 month, at least about 2 months, at least about 3 months, at least about 4 months, at least about 5 months, or at least about 6 months.

In certain embodiments, the soft tissue device is stable at a temperature of 37° C.

The soft tissue device of implant may be used for implanting or injecting administration around the target tissues.

Fiber-Hydrogel Composites Combined with Adipose Tissue

Provided herein are nanofiber-hydrogel composites combined with adipose tissue (composite-adipose) for use medical devices that are incorporated into a tissue of a human subject to whom the complexes are administered, e.g., by injection or implantation. Composite-adipose can be prepared by any means known in the art. Different ratios of adipose and fiber-hydrogel composites may be combined to obtain optimum results for the desired outcome. In preferred embodiments, the adipose tissue is lipoaspirate.

In certain embodiments, nanofiber-hydrogel composites may be gelated for a certain period of time such as about 1 hour, 3 hours, 5 hours, 8 hours, 12 hours, 24 hours, 36 hours, 48 hours, or from about 1 hour to 24 hours, prior to adding the adipose tissue. In certain embodiments, the nanofiber-hydrogel composites are gelated and stored (in hydrated or lyophilized form) for an indefinite amount of time, prior to adding the biologicially active component, such as adipose tissue.

After the hydrogel composites (microbeads) and lipoaspirate are combined, superior mechanical integrity may be obtained as compared to lipoaspirate alone. The microbeads and lipoaspirate can be administered (e.g. injected) into a human subject in order to promote fat graft retention and vascularization, serving as a tissue scaffold that mimics native extra-cellular matrix. The approach offers promise for larger volume reconstruction without risks of implant failure and fibrosis. The composite-lipoaspirate has enhanced mechanical properties which are more similar to in vivo fat, compared to the currently clinically used processed lipoaspirate.

Depending on the adipose (e.g. lipoaspirate) to composite ratio, the storage modulus (G′) of the combined materials can be increased in a synergistic manner. This is important since the storage modulus is a measure of how deformable a material is, and the ideal tissue scaffold would have similar properties/strength as native adipose tissue. With higher storage moduli and a lower tan-delta, the adipose/composite combination is less deformable and thus stronger and less susceptible to shear forces. As such, having a fat-composite combination with a high storage modulus may relate to to improved adipocyte and fat graft survival in-vivo. This has strong clinical implications, as this translates to less morbidity and fewer procedures for the patient undergoing fat grafting for soft tissue reconstruction.

Composite-adipose have the ability to demonstrate superior rates of angiogenesis and blood vessel ingrowth as compared to lipoaspirate. An adequate blood supply is essential for fat graft survival. This why increased angiogenesis and blood vessel ingrowth provided by composite-adipose is necessary for improving long-term adipocyte survival. In addition, the composite-adipose does not deter adipocytes from accessing cell culture media for survival. Composite material provide adipocytes access to angiogenic growth factors required for long-term survival. On the other hand, lipoaspirate alone having a lipid layer surrounding the sample, thus preventing adipocytes from accessing cell media.

### Methods of Use

Provided also are methods of fat grafting in a subject by implanting or injecting the soft tissue device or implant as described herein.

The disclosure further provides methods for performing a cosmetic procedure or a reconstructive procedure or reducing or reversing a tissue defect resulting from trauma, surgical intervention, or an age-associated disease, disorder or condition. The methods include implanting or injecting the soft tissue device as described herein into a target tissue of the subject. Preferably, the biologically active material is capable of at least one of i) recruitment of host cell infiltration, ii) promotion of tissue growth, iii) and/or cell or tissue regeneration in a subject, and wherein the soft tissue device is capable of being implanted or injected into a target tissue of a subject in need thereof.

Further, provided are kits for preparation of the injecting the soft tissue device as described herein for administration into a target tissue of a subject. For example, the hydrogel composites (microbeads) and the biologically active material may be injected simultaneously or separately to the targe tissued of a subject in need thereof. In certain embodiments, the kit may include (i) a first syringe containing the microbeads; and (ii) a second syringe containing the biologically active material, wherein the microbeads is gelated or cured before the biological active material is added to the microbeads. In certain embodiments, the microbeads may be pre-reacted and lyophilized. In certain embodiments, the microbeads and the biologically active material (e.g., fat or fat tissue) are not gelated in-situ. For example, the biologically active material (e.g., fat or fat tissue) for fat grafting may be applied on or inside the hydrogel composite, which is pre-reacted/beaded.

In some embodiments, the kit further includes an additional syringe or vial containing water, saline solution, or suitable fluid for reconstitution of the dehydrated microbeads.

Although particular examples and uses for the hydrogel/nanostructure composites of the invention have been described herein, such specific uses are not meant to be limiting. The hydrogel/nanostructure composites of the invention can be used for any application generally used for known hydrogels, and in particular, are useful for the repair and/or regeneration of soft tissue anywhere in the body.

### EXAMPLES

The present compostions, methods and systems include technical refinements for enhanced survival and function of transplanted fat by mixing with fiber-hydrogel composites. Fat grafting is a treatment for restoration of soft tissue and volumization after loss of soft tissue due to trauma, surgery, disease, or ageing, with about 600,000 fat grafting procedures in the face worldwide in 2019, and substantially more fat grafting procedures in other anatomical regions [ISAPS 2019 International Survey]. However, fat grafting has limitations, as graft survival is variable and incomplete, from 34% to 82% in breast and 30-83% in the facial area [Yu, Nan-Ze et al. “A systemic review of autologous fat grafting survival rate and related severe complications.” Chinese medical journal vol. 128,9 (2015): 1245-51]. The results in animal studies can be even more stark, with survival rates of 38.3-52.5% after 15 weeks in nude mice and 14.00-14.56% after 1-year in rabbits [Yu, Nan-Ze et al. “A systemic review of autologous fat grafting survival rate and related severe complications.” Chinese medical journal vol. 128,9 (2015): 1245-51]. This impedes surgical precision, patient satisfaction, and may necessitate repeated surgical procedures.

The present compositions, methods and systems improve fat graft survival and volumization by mixing the processed fat with a fiber-hydrogel composite gel immediately prior to injection. In an aspect, this gel inclusion mimics the body's own extracellular matrix structure and thus provides mechanical support to the processed fat globules, protection from shear forces during the injection procedure, a lower tan delta (and hence more solidlike character) to resist unintended implant migration, adhesion sites for the cells after implantation, immunomodulation and protection for the transplanted cells, and angiogenesis to support the survival of the grafted cells. In an aspect, the form of the fiber-hydrogel composite of this invention is that of fragmented lengths of individual polymeric fibers dispersed evenly throughout the hydrogel phase, with interfacial bonding between the fiber and hydrogel phases. The gel is fully reacted and broken up into individual microbeads approximately 150 microns in diameter prior to mixing with the processed fat. By combining the fat with the composite, better outcomes can be achieved than the fat grafting alone.

Such combinations of fat with the fiber-hydrogel composites have been described in previous disclosures, but this is the first disclosure with the pre-reacted “beaded” form. Previous work was performed with an in-situ gelling form of the composite. The use of the beaded form of the composite gel in this disclosure highlighted different optimal ratios of concentrations and sizes of the two components (microbeads of fiber-hydrogel composites and fat particles), leading to enhanced soft tissue regeneration.

### Example 1: Nanofiber-Hydrogel Composite Beads Preparation

The nanofiber-hydrogel composite (lot 0026-093020-1) was produced using the methods described in the patent application PCT/US2019/031638. Briefly, the gel consists of polycaprolactone (PCL) fiber fragments dispersed evenly within a hyaluronic acid (HA) hydrogel, with polyethylene glycol dithiol (PEG-SH) as a crosslinking agent. The PEG-SH reacts with acrylate groups functionalized on the HA molecules and with maleimide groups on the PCL fiber surface, forming a three-dimensional network hydrogel with covalent-strength interfacial bonding between the hydrogel phase and polymer fiber phase. The concentration of PEGSH (5 kDa molecular weight) was set for a molar stoichiometry of 1:1 between the thiol groups of the PEG-SH and the acrylate groups of the HA (9% acrylation degree) to maximize crosslinking and minimize pendant crosslinking groups. The composition was 8 mg/mL HA, 30 mg/mL PCL fibers, 4.6 mg/mL PEG-SH, and 3 mg/mL lidocaine hydrochloride. The gel was formulated in phosphate-buffered saline (DPBS) and fully reacted, then broken up into non-spherical beads of approximately 150 microns in size in order to be injectable.

### Example 2: Rodent Fat Processing and Preparation of NHC-Fat Grafting Materials

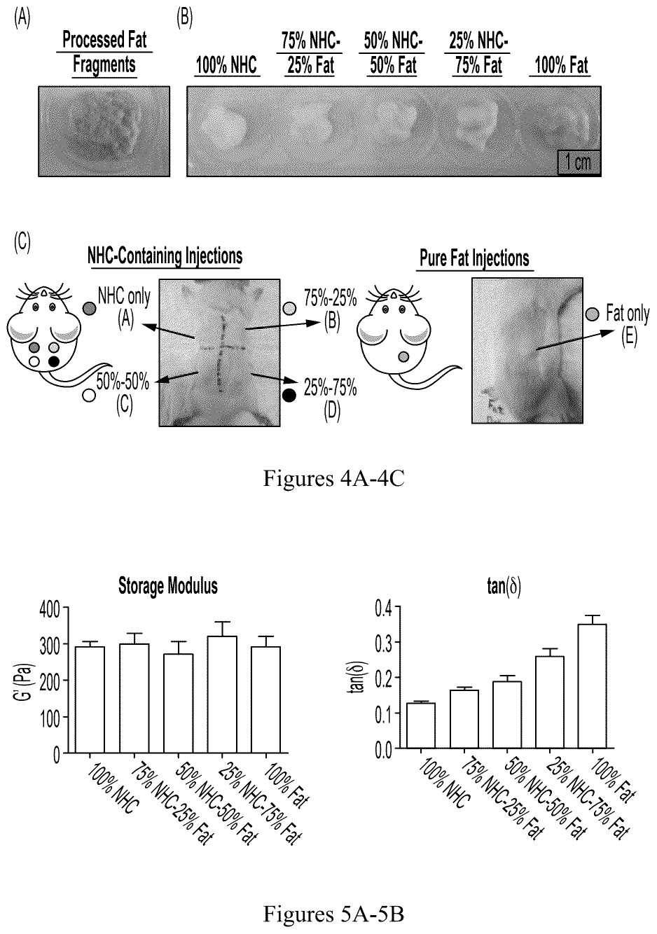

Intact adipose tissue was extracted from the fat pad in the inguinal regions of female Lewis rats (approximately 10 cc collected per rat). The collected tissue was minced into small fragments using a surgical scissor. Three milliliters of the minced fat were then transferred to and evenly spread on a piece of Telfa™ pad to remove the oily components. A total of two Telfa™ treatments was applied to the fat fragments. The processed fat as shown in FIG. 4A was collected in syringes and was ready to be mixed with the NHC of example 1.

The fat may be further processed to form fat particles as demonstrated in Example 3. The fat for each experiment was pooled together and mixed before being aliquoted for each NHC mixture group. Approximately 20 cc of fat from two rats was pooled for the mechanical testing and approximately 40 cc from 4 rats was pooled for the in vivo study.

Three different combinations of the NHC and the processed rodent fat were prepared by manually mixing the two parts in syringes at designated volumetric ratios (Table 1 and FIG. 4B). Two control groups-pure NHC and pure fat samples-were also included.

TABLE 1
Combinations of the NHC and the Processed Fat for Fat Grafting

| | (A) Pure | (B) 75N- | (C) 50N- | (D) 25N- | |
|---|---|---|---|---|---|
| Group | NHC | 25F | 50F | 75F | (E) Pure Fat |
| NHC | 100% | 75% | 50% | 25% | 0% |
| Fat | 0% | 25% | 50% | 75% | 100% |

The prepared grafting materials were then examined in an ARG2 rheometer to determine the storage moduli (G′). Briefly, a sample was first trimmed into a disc of 8-mm in diameter by 2-mm in thickness sandwiched between two parallel plates. An amplitude sweep test with the strain ranging from 0.01 to 10% (at 1 Hz) was applied to identify an optimal amplitude value for the sample, followed by a frequency sweep test (0.1-10 Hz at 1% strain). The storage modulus was calculated by averaging the values within the linear range of the frequency sweep curve. The tan delta values (loss modulus divided by storage modulus) were calculated over the same range.

### Example 3: Fat Particle Formation

Animal fat (e.g., fish oil or rat fat) was thickened using heat and oxygen until the desired viscosity is reached. The thickened oil is cast onto PTFE coated plates using an adjustable casting knife set and UV treated, and then heated to obtain a cross-linked, fatty acid-derived biomaterial film. The film can be converted into particles either by placing the film into a mortar, covering it with liquid nitrogen and using the pestle to grind it into particle form or by cryogrinding it into particle form.

Alternatively, fat may be obtained from a mammal (e.g., rat or human) by using aspirator (e.g., vacuum pump) and the obtained fat may be harvested using homogenizer. The collected fat samples may be filtered and the filtration may be controlled based on the desired particle size. It is appreciated that the REVOLVE™ (Abbvie) may provide the apparatus for preparing the fat particles.

Alternately, fat tissue can be surgically harvested en bloc from a donor site (either from the same animal/person or from a donor animal/person), then mechanically macerated to generate fat particles with intact cells in extracellular matrix.

### Example 4: Fat Grafting and Graft Assessments

The prepared grafting materials from Example 2 (500+50 μL per injection) were subcutaneously injected on the back of each Lewis rat using disposable 14G intravenous catheters. Each rat received up to four grafts and the injection schemes were summarized in FIG. 4C. The pure fat injections were performed on different animals separate from the NHC-containing samples to avoid systemic inflammation that may affect the outcome of the pure fat grafts.

Magnetic resonance imaging (MRI) was performed immediately after the injections and every 15 days thereafter to individually trace the volume of each injected graft over a time course of 90 days. The volume retention was determined by normalizing the volume of each sample at a specific time point to its corresponding initial value at day 0 (post-operative day or POD 0).

The injected grafts were harvested at PODs 0, 30 and 90 for histological and immunofluorescent analyses. An initial evaluation of graft morphology was done by hematoxylin and eosin staining, followed by immunolabelling with (i) perilipin and (ii) α-smooth muscle actin (αSMA) and rat endothelial cell antigen (RECA-1) to visualize viable adipocytes and newly formed blood vessels, respectively.

### Results

### Comparable Storage Moduli and Uniform Graft Morphologies Across Different Combinations of the NHC and the Fat Fragments

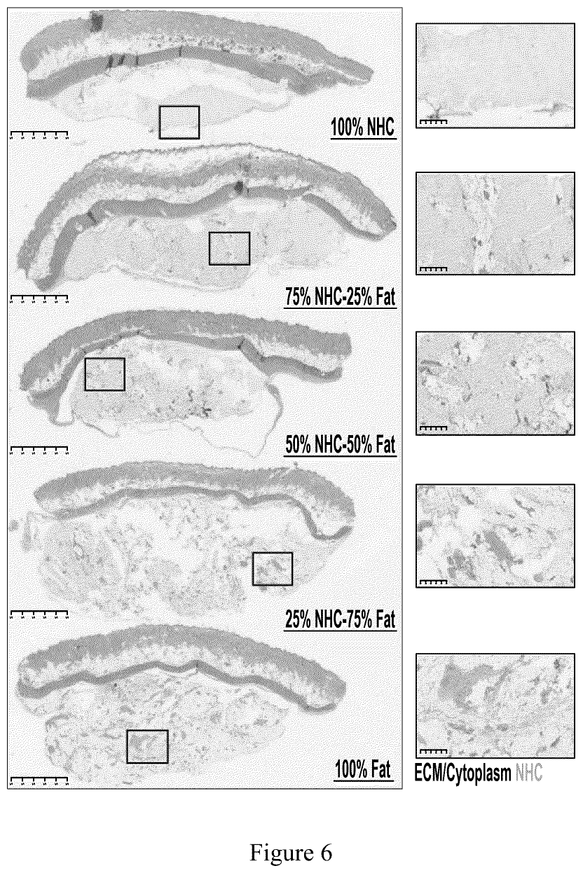

The NHC and the processed fat fragments were mixed at different volumetric ratios and the prepared grafting materials were subjected to mechanical testing. The rheological results suggest that both the NHC and the processed fat exhibited similar mechanical strength with a G′ value of around 300 Pa. Therefore, when combined together at different percentages, the resulting storage moduli were comparable across different groups as shown in FIG. 6. The tan delta values were lower for the composite gel (0.13) than the fat (0.36), with the mixtures having intermediate values correlating with their relative compositions (0.17 for 75% composite, 0.19 for 50% composite, and 0.27 for 25% composite). This demonstrates that mixing processed fat with the fiber hydrogel composite can be used to reduce the tan delta of the mixture, making it more “solidlike” to better maintain its intended implantation shape and resist flow or migration after injection.

The injected grafts were harvested immediately post-injection to evaluate the distribution of the NHC and the processed fat under different mixing conditions. Our H&E staining results (FIG. 6) demonstrate that despite imperfect homogeneity, the NHC and the processed fat were evenly distributed within each type of graft, indicating a good mixing between the two components.

50% NHC-50% Fat Grafts Achieved Superior Volume Retention after 3 Months In Vivo in Comparison with the Other NHC-Fat Combinations

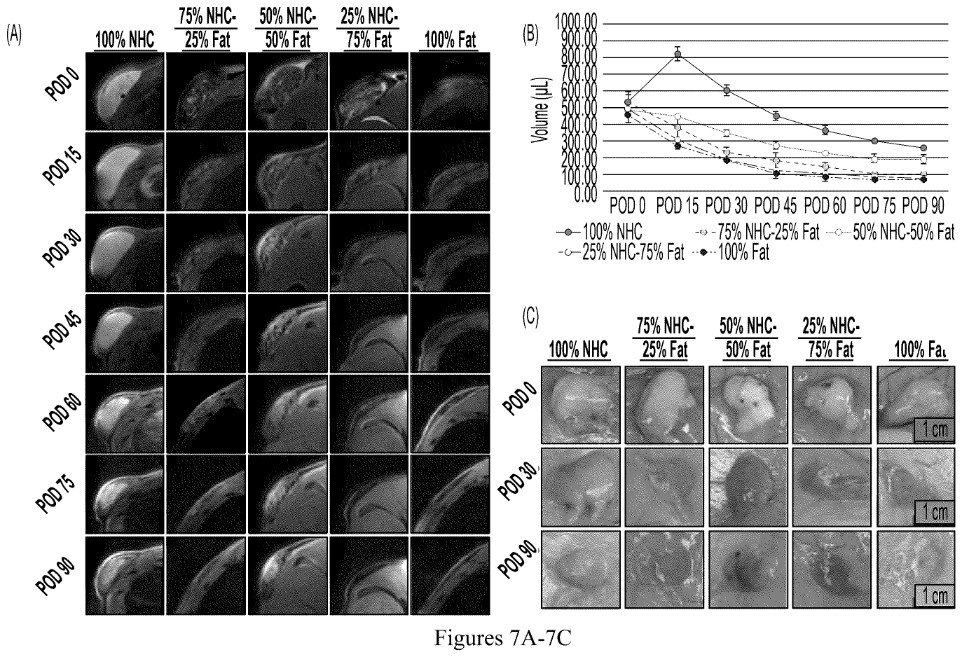

MRI was performed over a time course of 90 days (FIG. 7A) to determine the volume and shape retention of each injected graft over time. Specifically, graft swelling was only observed in the pure NHC group within the first two weeks post-injection whereas the volume of all the other types of grafts continued to decrease over time from POD 0 (FIG. 7B). After POD 45, the graft volume in most of the groups began to stabilize with an exception of the pure NHC injection, yet its volumetric decreasing rate was also slowing down. By POD 90, the 100% NHC grafts exhibited the best volume retention followed by the 50% NHC-50% Fat group which demonstrated 49.49±3.77% and 40.37±6.27% of their initial values, respectively (FIG. 7B and Table 2), and the difference between the two groups was not statistically significant (p=0.056). Conversely, the volume retention of all the other types of grafts was significantly compromised and was no more than 20% when compared to the corresponding POD 0 values.

TABLE 2
Volume Retention of Injected Grafts at POD 90, n = 4.
Volume Retention (V_{POD 90} to V_{POD 0})

| | (A) Pure | | | | (E) Pure |
|---|---|---|---|---|---|
| Group | NHC | (B) 75N-25F | (C) 50N-50F | (D) 25N-75F | Fat |
| % | 49.49 ± | 20.34 ± 1.63 | 40.37 ± 6.27 | 17.16 ± 4.23 | 16.63 ± |
| | 3.77 | | | | 3.52 |

Moreover, based on the MRI and gross images (FIGS. 7A and 7C), the injected grafts showed a bolus-like shape at POD 0 in all the groups, but got flattened out over time especially in 75% NHC-25% Fat, 25% NHC-75% Fat, and 100% Fat groups. Furthermore, the pure fat grafts continued to be absorbed by the host and became less visible by POD 90. Abundant vacuoles and viable adipocytes remained in 50% NHC-50% Fat grafts after 3 months in vivo

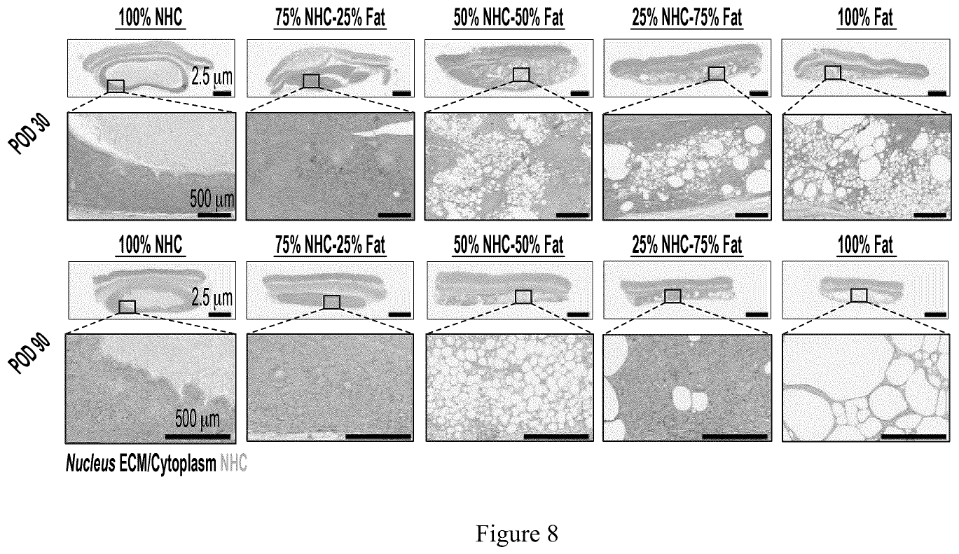

Morphologically, the pure NHC grafts developed into a capsule-like structure in which an NHC core was surrounded by an outer cellular ring that was in close proximity to the host tissue and cells such that tissue remodeling into neo-soft tissue first occurred in this region and gradually progressed toward the center of the construct (FIG. 8). The 75% NHC-25% Fat grafts contained few or virtually no vacuoles, a unique morphology owned by native adipose tissue, at POD 30, indicating early tissue remodeling in this group and by POD 90, these constructs completely remodeled into a soft tissue-like structure. A hybrid composed of both adipose tissue and remodeled soft tissue was observed in the other three groups (50% NHC-50% Fat, 25% NHC-75% Fat, and 100% Fat) at the early stage of the fat grafting process. However, after 90 days in vivo, the 50% NHC-50% Fat combination was the only group which had abundant vacuoles with a proper or healthy size ranging between 50 to 100 μm whereas the majority of the 25% NHC-75% Fat grafts remodeled into soft tissue with few enlarged vacuoles and the pure fat injections contained extremely large vacuoles.

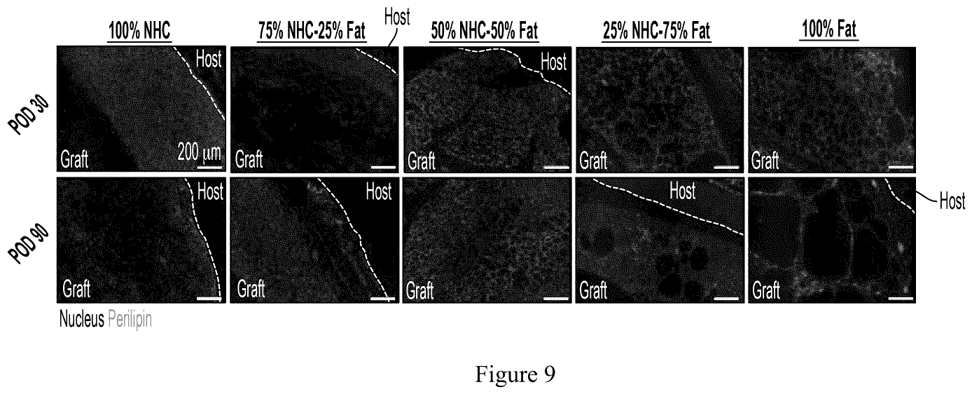

The presence of viable adipocytes within each type of graft was detected by staining the specimens for perilipin, a lipid droplet-associated protein, and a non-viable adipocyte is defined as a cell that has an enlarged vacuole-like structure with no or little perilipin staining and is usually surrounded by infiltrating host cells (FIG. 9). Specifically, no perilipin staining was observed in the pure NHC and the 75% NHC-25% Fat grafts at POD 30, yet some viable adipocytes were found in the peripheral area of the remodeled tissue in both groups at POD 90 that were not shown at the earlier time point. Since these adipocytes were quite close to the boundary with the host, they were very likely to be migrating from the host to the injected grafts. On the other hand, there were a lot of non-viable or dying adipocytes detected in both 25% NHC-75% Fat and 100% Fat groups at the early stage of the fat grafting process. By POD 90, the 25% NHC-75% Fat samples contained little perilipin staining while the pure fat grafts consisted mainly of non-viable adipocytes. Noteworthily, the 50% NHC-50% Fat constructs displayed a mixed morphology of abundant viable adipocytes and remodeled soft tissue at both early and late time points. This finding suggests that the 50% NHC-50% Fat combination may offer a superior condition that can maintain or improve the survival of the processed fat co-injected with the NHC as validated by the increased viability of adipocytes within the grafts.

a Higher Degree of Vascularization was Achieved in 50% NHC-50% Fat Grafts after 3 Months In Vivo

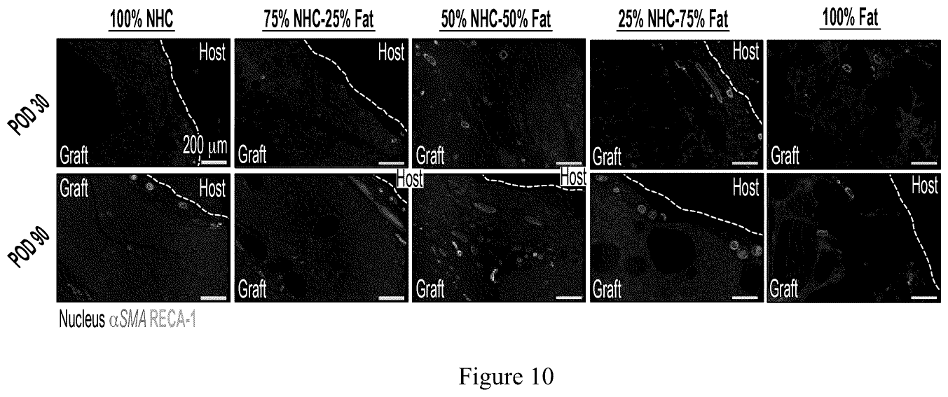

The grafts were harvested and co-stained for αSMA and RECA-1 to detect blood vessel formation within each type of graft (FIG. 10). Our data demonstrate that elevated vascularization was observed in the 50% NHC-50% Fat constructs in comparison with the other fat-containing samples, which potentially contributes to the improved survival of the co-injected fat fragments in this group. Conversely, the pure fat grafts remained poorly vascularized throughout the 90-day fat grafting process. Furthermore, while most of the blood vessels were identified at the edge of the construct in 100% NHC, 75% NHC-25% Fat, and 25% NHC-75% Fat groups, some small vessels were also found toward the interior of the remodeled soft tissue by POD 90. However, their overall degree of vascularization was still inferior to that of the 50% NHC-50% Fat group.

### CONCLUSION

The pure NHC injections led to the highest volume retention after 90 days in vivo, and the injected grafts developed into a core-shell-like structure with an NHC core and an outer cellular ring of slowly infiltrating cells and remodeling.

The 75% NHC-25% Fat grafts underwent rapid soft tissue remodeling such that only few or virtually no fat fragments remained in the constructs at POD 30 and completely developed into soft tissue with few viable adipocytes detected at the periphery by POD 90. The composite gel component was degraded much more rapidly than in the 100% NHC group, and the volume maintenance was considerably lower than the 100% NHC and 50% NHC groups.

The volume retention in the 50% NHC-50% Fat group was slightly lower than the 100% NHC group at the end of the fat grafting process, but the two values were not significantly different. This may result from increased vascularization of these grafts that potentially contributes to improved survival of the fat fragments co-injected with the NHC and further to better volume retention when compared to the other fat-containing constructs. The 50% NHC-50% fat group had excellent cellular infiltration, promoted the survival or growth of mature adipocytes throughout the implant to a far greater extent than any other group, and had a greater density and distribution of blood vessels.

Although the 25% NHC-75% Fat grafts displayed a mixed morphology of adipose tissue and soft tissue at the early stage of the fat grafting process, most of adipocytes in the constructs were non-viable or dying such that by POD 90, the majority of the grafts remodeled into neo-soft tissue with few enlarged vacuoles.

The pure fat grafts were poorly vascularized at any time points leading to a compromised viability of injected adipocytes and poor volume retention.

Benefits from Beaded Form

The performance of these groups are superior to the gels of the previous patent filing that discussed the combination of fat with composite hydrogel (US 2020/00304996). The previous form of composite gel used with fat grafting were not in the microbeaded form. The form was that of gel constituents that were mixed together (and with the processed fat) before they had gelled. The mixture was then injected in the partially-gelled state (after incubation for 3 hours at 38C in example 18) required in-situ gelation, and so was mixed with the processed fat in a pre-gelled condition for in situ gelation in the body after injection.

### Irregular Shape and Porosity for Reconstruction/Shape Retention

The microbead morphology is an important distinguishing trait from the prior art. The Martin et al filing described a continuous, intact gel, that would not be injectable (the intact gel, taking on the dimensions of the syringe, is much larger in dimension than the inner diameter of the syringe needle, such that the gel has to be broken up by the process of being pushed through the needle, which massively increases the force required). Other particulate-based gels in this product category such as Radiesse, Sculptra, or Ellanse are composed of spherical non-porous particles in a gel carrier, and the smooth spherical surface is desired to achieve the mechanism of action (durable volumization caused by collagen deposition on the particle surface). The smooth spherical design minimizes the surface-area to volume ratio, which is important because increasing surface area of non-porous materials in the body is associated with an undesired inflammatory foreign body response by the body's immune system.

The mechanism of action of those devices is different in the current invention, which achieves durable volumization by acting as a substrate or sponge that encourages cellular infiltration into the microbeads. Since the microbeads themselves are porous, the surface of the beads do not cause the same immune response and the surface area to volume ratio does not need to be minimized. The irregular, non-spherical microbead shape thus has a higher surface area to volume ratio, which increases the microbead-microbead contact area, increasing the desired cohesiveness of the injected gel. The individual microbeads of this composition also have a plurality of fibers present at every surface of the microbead, creating more friction and mechanical entanglement at the interface of two microbeads. The microbead gel particles readily stick to one another, so increased bead-bead contact area makes gels that better maintain their shape after injection, feel more solidlike to better mimic the natural soft tissues, resisting forces from surrounding tissue that could cause gel migration away from the intended area of augmentation. Indeed, a decrease in tan delta when a bulk gel was formed into microbeads (both 150 and 250 micron sizes), showing that the microbeads can be more solidlike than the bulk gel. The irregular, non-spherical shape of the flexible, porous microbeads also allows more efficient packing compared to spherical beads in a carrier (fewer voids in between particles), which likewise improves gel cohesiveness. The size, shape, and flexibility of the beads allows for good mixing with the biologically active material (e.g. adipose tissue), with gel material generally within a few hundred microns of the implanted tissue at all points. This provides mechanical support to the tissue to protect it during implantation and prevent migration after implantation, and generates the necessary blood supply over longer timepoints to keep the implanted tissue alive (since the gel phase can encourage angiogenesis).

### Potential Mechanisms for the Importance of Volume Ratios

Processed fat has challenges for use in the implantation process—the fat cells are fragile (prone to adipocyte lysis due to shear forces during liposuction, processing, and implantation), and has relatively high tan delta (more “liquidlike” and thus prone to spreading/migrating in response to pressures from the surrounding tissue after implantation, limiting surgeon control over placement and ultimately minimizing the intended volumizing effect). Once implanted, generally only approximately 50% of the fat volume is maintained over time due to many factors, including localized ischemia due to a lack of blood supply and inflammation or clearance from the immune system. When the adipocyte cells die (either due to mechanical forces, inflammatory response, or ischemia) they can release oil that can cause inflammation and phagocytic degradation as opposed to remodeling. This leads to oil cysts that ultimately lead to little durable volumization and tissue remodeling (such as angiogenesis and ECM deposition). This is seen in the poor volumization, large vacuoles, and minimal remodeling through 90 days in the 100% fat group. The Composite hydrogel by itself is effective at maintaining volume but doesn't fully recapitulate the native subcutaneous tissue. This is seen in the 100% NHC group, with little adipogenesis, and slower rate of angiogenesis, with only part of the implantation site remodeled after 90 days. Combining the processed fat together with the composite gel enhances the gel response by providing adipocytes, supporting cells, native fat structures and growth factors. Combining the processed fat with the composite gel enhances the fat graft response by providing mechanical support, shear protection, sites for cell adhesion, immunomodulation to limit inflammation or a phagocytic macrophage response, a pro-regeneration polarization of macrophages, enhanced angiogenesis for limiting ischemia, and a lowered tan delta to improve volumization and resist implant migration in the body. The ideal ratio of fat to composite gel is not clear so as to simultaneously maximize volume maintenance, adipocyte proliferation and survival, and angiogenesis. Too little composite (such as 75% fat, 25% composite) may be insufficient to prevent the negative cycle of necrosis, oil pooling, inflammation. Too little fat (such as 25% fat, 75% composite) may also be non-ideal, as the rate of angiogenesis through the gel phase may be too slow to prevent ischemia and necrosis for the implanted cells in the center of the injected fat-gel implant, leading to the same necrosis negative cycle.

### Potential Extensions

These examples used allogenic fat transfer, due to the small volumes available in the rat model. The minimal manipulation of fat required in this procedure makes it a natural candidate for autologous fat transfer. The fat would be removed from the patient (such as by liposuction), processed, mixed with the composite gel, then injected back into the patient. The transferred cells and/or tissue can consist of whole fat transfer, the Stromal Vascular Fraction (SVF), or stem cell transfer (mesenchymal stem cell or adipose stem cells) either harvested from the patient or from a cell bank.

The composite gel can be made from formulations with increasing or decreasing constituent concentration or alternative fiber forms (such as collagen or cellulose fibers) or alternate hydrogel phases (either with a different crosslinker like BDDE or DVS, or a different gel-forming molecule like collagen, PEG, carboxymethyl cellulose, chitosan, alginate, chondroitin sulfate, PVA).

The fat survival and implant function could be further modulated by incorporating drugs. These can be selectively tuned for burst release (soluble, unencapsulated drugs) sustained release (drugs loaded into hydrogel phase nanoparticles or drugs co-spun into the fibers, or drugs covalently linked into hydrogel phase), or drugs surface-bound to the fibers through covalent bonding or another method. Such bound drug presentation can have different biological effects vs a freely soluble form (as seen with VEGF). The included drugs could consist of growth factors (e.g., bFGF, IGF-1, PDGF, VEGF, HGF), vitamins, antioxidants, anti-inflammatory agents, nutraceuticals/extracts, pain relievers, drugs, surfactants (such as Pluronic F68), or others.

### Example 5: Collagen Fiber Production

Bovine source type I collagen solution was purchased from Advanced Biomatrix. Bovine collagen solution was firstly lyophilized overnight to obtain collagen powders, and then type I collagen solution (8 w/v %) was prepared in 1,1,1,3,3,3-hexafluoro-2-propanol (HFIP) at room temperature for around 6 hours to make a viscous cloudy electrospinning solution. The electrospinning was performed with the following parameters: 5 mL/h of the flow rate; 20-25 kV of the voltage applied to the 22-G metallic needle; 12.5 cm of the collecting distance; 900 rpm of the rotation rate of the metallic collector. This set of parameters results in a mean fiber diameter of around 600 nm. By using carbodiimide chemistry, fibers were immersed in ethanol solution (95% v/v %) containing 50 mM 1-Ethyl-3-(3-dimethylaminopropyl) carbodiimide (EDC) and 20 mM N-hydroxysuccinimide (NHS) for 24 hours. After the crosslinking, fibers were washed in 0.75% glycine solution three times with 5 minutes each time to remove the excessive reagents and to quench the activated fiber surface. The collagen fibers were then broken down to fragments using cryomilling (Freezer/Mill 6770, SPEX SamplePrep). The fragments were filtered through different cell strainers (40 and 100 μm) to reach a relative uniform fiber length.

### Example 6: Preparation of the Collagen Fiber-HA Hydrogel Composite

A preferred NHC construct comprises composed of three components: hyaluronic acid (HA) network, bovine type I collagen nanofibers, and divinyl sulfone (DVS) crosslinker). Before incorporating the collagen nanofibers (produced in Example 5 above) into the HA network during crosslinking, we optimized the crosslinking conditions for the HA gel phase alone.

Sodium hyaluronate (MW 1.5 MDa) was purchased from LifeCore. HUVECs and vascular endothelial cell culture medium were purchased from Lonza. All other chemical reagents were purchased from Sigma-Aldrich. All other cell culture reagents and supplements were obtained from Invitrogen. HA was dissolved in distilled water at a stock concentration of 25 mg/mL. DVS concentration was calculated as the ratio to the hydroxyl groups in HA (such as 1.17 w/v %, 2.34 w/v %, and 4.68 w/v %). The stock HA solution was diluted to 2 w/v % using distilled water and sodium hydroxide to get four different pHs (12.4, 12.7, 13.0 and 13.3), with other parameters set to be the same (2w/v % HA, 37° C., 3 hour reaction time). The reaction time or gelation kinetics were performed by preparing multiple samples and measuring the mechanical properties at various timepoints (30 min, 1 hour, 2 hours, 3 hours, 4 hours, 8 hours and 16 hours) to get a timepoint where the stiffness reaches a plateau. The crosslinking of the NHCs followed the same conditions to the HA hydrogels, different fiber density (0, 1 and 3 w/v %) were added to the mixed precursors to test the gelation kinetics of the NHCs. After the gelation of hydrogels and NHCs, dialysis was performed using dialysis membranes (6000-8000 MWCO, Spectrum) against pH 7.4 phosphate buffer for 48 hours to remove the unreacted DVS, to balance the pH and to swell the samples for further studies. The mechanical properties were measured again after the swelling. Microgels were then generated with stainless steel wire cloth discs to reach a gel particle size at around 100 μm as we previously reported.

We found that using DVS chemistry to crosslink the HA network at a pH of 12.7 was effective in forming a robust crosslinked hydrogel while minimizing the degradation of HA molecules and collagen fibers during gelation. The reaction pH had a large effect upon the resulting gel in the range of pH 12-13.3. With other parameters set to be the same (37° C., HA concentration 2 w/v %, DVS concentration 2.93 w/v %), while the reaction pH was set to be 12.4, 12.7, 13.0 and 13.3 prepared by different NaOH concentrations (0.001 M, 0.01 M, 0.1 M, 1 M). The reaction at pH 13.3 and 13.0 showed a dramatic degradation after 2 hours of reaction, resulting in two unstable hydrogels with low reproducibility.

The crosslinking time was optimized to around 2 hours to reach the maximal storage modulus and limit degradation. After tuning the DVS chemistry, we introduced the nanofibers to the HA network while crosslinking to generate interfacial bonding between the HA network and the nanofibers, and a reinforcement effect was observed comparing HA and NHC at a similar crosslinking density. Additionally, the composite could pass through 27-gauge needle easily after crosslinking. To further investigate this reinforcement effect quantitatively, we measured the storage modulus GO′ (normalized to a storage modulus control) of the HA hydrogel phase, the G′ of the overall NHC and the G′ of a hydrogel-nanofiber mixture without interfacial bonding. In a rheological test with 1 to 3 w/v % of fiber loading density, the G′ of the composite was ranging from 1.5- to 4-fold higher than that without interfacial bonding, and the G′ difference increased with the increase of the fiber loading and the crosslinker concentration. Furthermore, we also investigated the effect of fiber lengths on the stiffness enhancement by using different cell strainers (40 μm, 100 μm, unscreened) to screen out the large fiber fragments. In an unintuitive result, the gels with the fiber fragments with length between 40 μm to 100 μm helped generate the largest stiffness enhancement, though this relative enhancement was minimized at the highest crosslinking concentrations.

In consequence, these tuning steps allowed us to generate NHC with G′ in the range of 450 Pa to 1500 Pa after crosslinking, and 150-Pa to 1000-Pa after swelling. To mimic the soft tissue microenvironment, HA controls (G′=100-Pa and G′=250-Pa) and composite (G′=250-Pa and GO′=100-Pa) were generated and particularized to microgels with diameters around 100 μm. The storage modulus for all three groups were measured not significantly different from the initial crosslinked bulk gels. Lastly, before utilizing the prepared materials for later in vitro and in vivo studies, we autoclaved the hydrogels and composites and observed that the G′ measurements were not significantly lowered after the terminal sterilization, indicating the translational potential of this material. Autoclaving was performed to sterilize the hydrogels and NHCs after gelation. Briefly, the gels were placed at the autoclave cycle at 118° C. with a 5-minute sterilization step. The total sterilization cycle would take 30 minutes to complete. After the sterilization, the mechanical properties of each gel were measured again using rheological tests. The thermal stability during autoclaving also indicates that the gels will have excellent shelf stability at the much lower temperatures required for ambient storage.

### Example 7: Fat Grating by Co-Injecting of Process Adipose Tissue Particles with Collagen Fiber-HA Hydrogel Composite (Matrix C)

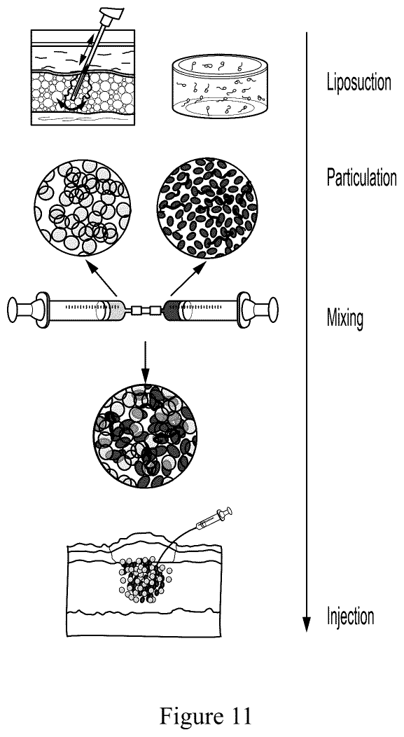

The prepared grafting materials composed of different volume ratios of the collagen fiber-HA hydrogel composite of the type prepared in Example 6 above (this collagen fiber-HA hydrogel composite referred to in this Example as Matrix C) and processed fat particles at a total volume of 500±50 μL per injection, were subcutaneously injected in the back of Sprague Dawley (SD) rats using a disposable 14-G intravenous catheters (FIG. 11). The injected grafts were harvested on day 30 after implantation for volumetric, histological, and immunofluorescent assessments. The volumetric analysis was based on gross images and caliper measurements. The histological evaluation includes hematoxylin and eosin staining, followed by immunofluorescence imaging with perilipin to detect viable adipocytes.

### Results:

Matrix-Co-Injection Improved Volume Retention of the Fat Graft In Vivo after 1 Month

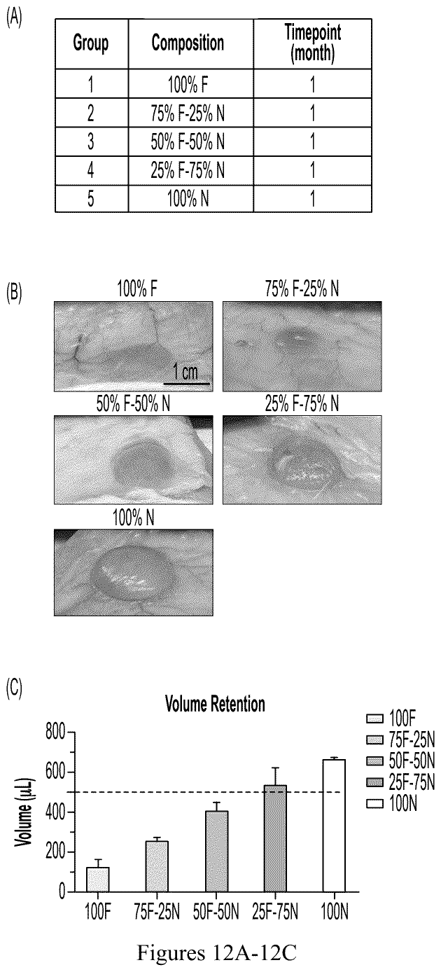

After sacrificing the rats on day 30, gross image measurements based on calipers were performed to determine the volume and shape retention of each injected graft over time. Specifically, with the increasing volume fraction of Matrix C, the retention volume of the fat tissue also increased (FIG. 12), although the best volume retention was achieved with 100% Matrix C injection, likely due to the slow degradation of the hydrogel material. This demonstrates that the inclusion of Matrix may moderately increase the survival fraction of the fat grafts.

### Superior Adipocyte Survival was Observed Between 75% Matrix C/25% Fat Graft and 50% Matrix C/50% Fat Graft

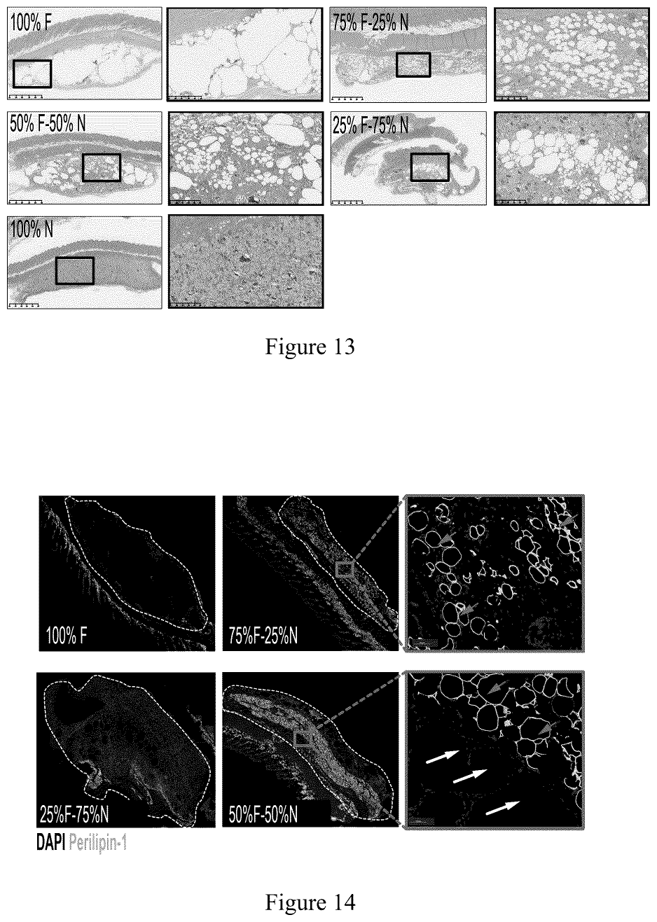

The grafts were harvested and stained for hemoxylin and eosin for histological analysis. Specifically, on day 30, pure fat grafts showed the enlarged vacuoles at the subcutaneous space, indicating the necrosis of adipocytes. Pure Matrix C showed host cellular infiltration, vascularization, and signs of neo-soft tissue formation. Both 50% Matrix C-50% fat and 25% Matrix C-75% fat maintained a portion of adipocytes with no enlarged vacuoles, but the number of those healthy adipocytes were limited. The 25% Matrix C-75% fat was the only group at this timepoint which had abundant adipocyte vacuoles with a healthy size range, and they distributed across the injected grafts (FIG. 13).

To furthermore detect and differentiate viable and non-viable adipocytes, perilipin-1 staining was performed to mark a lipid droplet associated protein which is usually presented in viable adipocytes (FIG. 14). Specifically, by day 30, both 25% Matrix C-75% Fat and 50% Matrix C-50% fat grafts showed a superior adipocyte survival. However, in 50% Matrix C-50% fat grafts, some enlarged vacuoles with no perilipin expression were observed at the periphery, indicating the presence of non-viable adipocytes. Beyond the range between these two compositions, very limited viable adipocytes were observed in other tested materials. This finding suggests that the compositions between 25% Matrix C-75% fat and 50% Matrix C-50% fat grafts may offer a superior microenvironment that can improve the survival of the processed fat fragments with the co-injection of the Matrix C.

### EQUIVALENTS

It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

1. A composition for administration, comprising:
fat cells or tissue; and
non-spherical microbeads comprising a hydrogel composite, the hydrogel composite comprising a hyaluronic acid component covalently linked to a fiber component, and
a weight ratio between the hyaluronic acid component to the fiber component ranges from 1:100 to about 100:1.

2. The composition of claim 1 wherein the fat cells or tissue are adipose cells or particulates of adipose tissue.

3. The composition of claim 1, wherein the composition comprises particles that comprise fat cells or fat tissue.

4. (canceled)

5. The composition of claim 2, wherein a ratio of the mean size of the non-spherical microbeads to a mean size of the fat particles ranges from about 2:8 to 8:2.

6. The composition of claim 2, wherein a ratio of the mean size of the non-spherical microbeads to a mean size of the fat particles ranges from about 3:7 to 7:3.

7. The composition of claim 2, wherein a ratio of the mean size of the non-spherical microbeads to a mean size of the fat particles ranges from about 4:6 to 6:4.

8. The composition of claim 2, wherein a mean size of the non-spherical microbeads is within 5 or 10% of a mean size of the fat particles.

9. The composition of claim 1, wherein the microbeads are gelated or cured before the biological active material is added to the microbeads.

10. The composition of claim 2, wherein the mean size of the fat particles is less than about 500 μm.

11. (canceled)

12. The composition of claim 1, wherein the hydrogel composite is formed by combining the functionalized hyaluronic acid, the polymeric nanofibers, and a crosslinking agent.

13. The composition of claim 12, wherein the functionalized hyaluronic acid comprises thiolated hyaluronic acid, and the crosslinking agent comprises poly(ethylene glycol) diacrylate (PEGDA), or a derivative thereof.

14. The composition of claim 12, wherein the functionalized hyaluronic acid comprises acrylated hyaluronic acid, and the crosslinking agent comprises thiolated poly(ethylene glycol), or a derivative thereof.

15. The composition of claim 1, wherein the polymeric fiber component comprises polycaprolactone fibers.

16. The composition of claim 1, wherein the polymeric fiber component comprises collagen.

17. (canceled)

18. The composition of claim 1, wherein the weight ratio between the hyaluronic acid component to the fiber component ranges from 20:80 to about 80:20.

19. The composition of claim 1, wherein the weight ratio between the hyaluronic acid component to the fiber component ranges from 30:70 to about 70:30.

20. The composition of claim 1, wherein the weight ratio between the hyaluronic acid component to the fiber component ranges from 40:60 to about 60:40.

21. The composition of claim 1, wherein the weight ratio between the hyaluronic acid component to the fiber component ranges from 45:55 to about 55:45.

22. The composition of claim 1, wherein the soft tissue device further comprises a compound selected from the group consisting of growth factors, compounds stimulating angiogenesis, immunomodulators, inhibitors of inflammation, and combinations thereof.

23. The composition of claim 1, wherein the soft tissue device further comprises one or more compounds that have therapeutic effects, vascularization effects, anti-vascularization effects, anti-inflammatory effects, anti-bacterial effects, antihistamine effects, and combinations thereof.

24-27. (canceled)

28. A composition for administration, comprising:
a) non-spherical microbeads comprising a hydrogel composite, the hydrogel composite comprising a hyaluronic acid component covalently linked to a fiber component, and a weight ratio between the hyaluronic acid component to the fiber component ranges from 1:100 to about 100:1, and
b) a population of adipose cells, autologous adipose cells, allogenic cells, adipose stromal vascular fraction, adipose tissue, autologous adipose tissue, lipoaspirate, a derivative thereof, or a combination thereof.

29. A method of fat grafting in a subject, comprising administering an effective amount of a composition of claim 1 to a subject in need thereof.

30. A method for performing a cosmetic procedure or a reconstructive procedure or reducing or reversing a tissue defect resulting from trauma, surgical intervention, or an age-associated disease, disorder or condition, comprising administering an effective amount of a composition of claim 1 to a subject in need thereof.

31-32. (canceled)

33. A kit for preparation of the injecting the soft tissue of claim 1 for administration into a target tissue of a subject, the kit comprising: (i) a first syringe comprising the microbeads; and (ii) a second syringe comprising the biologically active material, wherein the microbeads is gelated or cured before the biologically active material is added to the microbeads.

34-41. (canceled)
